(19) United States
(12) Patent Application Publication
(10) Pub. No.: US 2026/0061009 A1
(43) Pub. Date: Mar. 5, 2026

(54) METHODS OF TREATING DIABETES USING DEVICES FOR CELLULAR TRANSPLANTATION

(71) Applicant: Sernova Corporation, London (CA)

(72) Inventors: Philip Michael TOLEIKIS, Vancouver (CA); Amanda Susan MACGILLIVARY, London (CA); Delfina SIROEN, London (CA)

(21) Appl. No.: 19/182,487

(22) Filed: Apr. 17, 2025

Related U.S. Application Data
(63) Continuation of application No. 17/608,052, now abandoned.
(60) Provisional application No. 62/841,444, filed on May 1, 2019.

(51) Int. Cl. A61K 35/39; A61K 31/436; A61K 35/28; A61K 35/545; A61K 38/18; A61P 3/10

(52) U.S. Cl. CPC A61K 35/39; A61K 31/436; A61K 35/28; A61K 35/545; A61K 38/1866; A61P 3/10

(57) ABSTRACT

The present disclosure relates to methods of treating, preventing, or modulating diabetes in a patient in need thereof using a device for transplanting cells into a host body, specifically a method of treating diabetes in a patient in need thereof, comprising: implanting a device in the patient, wherein the device comprises: a porous scaffold comprising an immunologically compatible polymer mesh forming the walls of at least one chamber, an opening at either or both of a proximal end and a distal end of the chamber, at least one removable, non-porous plug configured to be positioned within the lumen of the at least one chamber, maintaining the device in the patient's body until the device is infiltrated with vascular and connective tissues; and infusing the chamber with cells, wherein at least some of the cells express insulin

The present disclosure claims the benefit of priority to U.S. Provisional Patent Application No. 62/841,444, filed May 1, 2019, which is incorporated herein by reference in its entirety.

The present disclosure relates to methods of treating a disease, disorder, or condition (e.g., diabetes) in a patient in need thereof using a device for transplanting cells into a host body. More specifically, in certain aspects, the present disclosure relates to methods of treating, preventing, or modulating diabetes (e.g., type 1 diabetes) in a patient in need thereof using a device for transplanting cells (e.g., islets of Langerhans cells) into a host body.

Recent discoveries in the field of cellular therapy present new opportunities for the use of cell transplantation in disease areas with critical, unmet medical needs. Currently, there are no fully effective drug therapies for many acquired and congenital diseases caused by loss of or damage to cells producing biomolecules necessary for control of physiological functions, such as diabetes (e.g., type 1 diabetes). Cellular therapy holds the promise of replacing lost or damaged cells with cells capable of improving the impaired physiological functions. Transplantation of islets of Langerhans cells, for example, is an attractive strategy for restoring carbohydrate control in patients with insulin-dependent diabetes.

Limiting factors in the application of cellular therapy include difficulty in transplanting cells into host tissue and ensuring that the transplanted cells continue to function without eliciting an immune response and/or causing other harmful side effects in the host. Attempts have been made to administer therapeutic cells directly into the host body, e.g., in the vascular system or by implantation in an organ or tissue. However, with direct cellular transplantation, the patient is often required to remain on life-long immunosuppressant therapy, and the immunosuppressant drugs can cause toxicity to the host and/or to the implanted cells. Additionally, direct exposure of the cells to blood may lead to an immediate blood-mediated inflammatory reaction (IBMIR) that initiates a coagulation cascade and/or can destroy a significant portion of the transplanted cells. Furthermore, cells may become lodged in microvessels and cause blockage and thrombosis of the vessels, which may result in a loss of function of the transplanted cells and/or damage to local tissue.

Another therapeutic approach is the delivery of cells using devices that provide a biologically suitable environment for the cells to reside in the host body. Challenges with this approach include poor incorporation of blood vessels into the device for nourishing the cells and maintaining an optimal environment within the device for long-term survival of the cells. In the absence of an immediately vascularized environment, transplanted cells are not able to obtain enough oxygen or easily eliminate wastes, and may rapidly die or become damaged through the effects of ischemia or hypoxia. Furthermore, even in situations where some vessels grow early on, the vessels may not be sustained. In addition, the natural inflammatory cascade of the body may also result in the death of or damage to cells. Some other difficulties encountered with this approach include excessive scarring and/or walling off of the device, incompatibility of the device material with the biological milieu, difficulties in imaging the device and the implantation environment, improper dimensions of the device affecting biological function of the cells, inability to load the appropriate number of cells for a sustained therapeutic effect, and difficulty in removing the device when it needs replacement. Furthermore, the device configuration may not be amenable to the external contours of the body, which can result in abnormal protrusions of the device, thereby making the device unacceptable to the patient from an aesthetic perspective.

Thus, there remains a need for effective therapies involving transplantation of cells into a host body.

In some embodiments, the present disclosure provides methods of using devices capable of delivering and/or maintaining cells in vivo for an extended period of time, while alleviating many of the problems associated with existing device-based cell therapy approaches. The present disclosure provides, in some embodiments, methods of treating a disease, disorder, or condition (e.g., diabetes) in a patient in need thereof. The present disclosure more specifically provides, in some embodiments, methods of treating diabetes in a patient in need thereof. An exemplary embodiment is a method of treating diabetes in a patient in need thereof comprising: implanting a device in the patient; maintaining the device in the patient's body until the device is infiltrated with vascular and connective tissues; accessing the implanted device; withdrawing a plug from the device; and infusing a chamber of the device with cells. Another exemplary embodiment is a method of treating diabetes in a patient in need thereof comprising: implanting a device in the patient; maintaining the device in the patient's body until the device is infiltrated with vascular and connective tissues; accessing the implanted device; and infusing a chamber of the device with cells. In some embodiments, at least some of the cells express insulin. In some embodiments, the cells and/or the insulin-expressing cells are administered at a borderline mass of about 3000 IEQ/kg or greater. In some embodiments, the cells and/or the insulin-expressing cells are suspended in blood (e.g., plasma and/or serum) from the patient. In some embodiments, the cells and/or the insulin-expressing cells are suspended in plasma from the patient. In some embodiments, the cells and/or the insulin-expressing cells are suspended in plasma from the patient, wherein the suspension of cells in plasma further comprises one or more growth factors. In some embodiments, the cells and/or the insulin-expressing cells are suspended in serum from the patient. In some embodiments, the cells and/or the insulin-expressing cells are suspended in serum from the patient, wherein the suspension of cells in serum further comprises one or more growth factors. In some embodiments, the blood, plasma, and/or serum contains one or more growth factors. In some embodiments, the blood, plasma, and/or serum (e.g., with or without one or more growth factors) contains one or more proteins that may provide nutrients to the cells, signal for improved vascularization and/or regeneration, and/or alleviate inflammation. In some embodiments, the device comprises: a porous scaffold comprising an immunologically compatible polymer mesh forming the walls of at least one chamber; at least one removable, non-porous plug configured to be positioned within the lumen of the at least one chamber; and at least one seal configured to enclose either or both of a proximal end and a distal end of the at least one chamber. In some embodiments, the device comprises: a porous scaffold comprising an immunologically compatible polymer mesh forming the walls of at least one chamber; and at least one seal configured to enclose either or both of a proximal end and a distal end of the at least one chamber. In some embodiments, the device comprises: a porous scaffold comprising an immunologically compatible polymer mesh forming the walls of at least one chamber. In some embodiments, the device comprises a coating, e.g., a biodegradable coating instead of or in addition to a removable plug. In some embodiments, the biodegradable material coats at least a part of the porous scaffold. In some embodiments, the coating is biodegradable and can temporarily isolate the chamber of the implanted device from host tissue.

In some embodiments, the porous scaffold has pores sized to facilitate growth of vascular and connective tissues around and through the walls of at least one chamber. In some embodiments, the porous scaffold comprises at least one chamber. In some embodiments, the porous scaffold comprises one chamber, two chambers, three chambers, four chambers, five chambers, six chambers, seven chambers, eight chambers, ten chambers, twelve chambers, or more chambers. In some embodiments, the porous scaffold comprises about eight chambers, about nine chambers, or about ten chambers. In some embodiments, the porous scaffold comprises multiple chambers that are connected laterally. In some embodiments, at least one chamber comprises an opening at either or both of a proximal end and a distal end of the chamber. In some embodiments, the proximal end and the distal end of the chamber are separated by a lumen that is bounded by the walls. In some embodiments, the at least one removable, non-porous plug extends along the lumen of the chamber. In some embodiments, the at least one removable, non-porous plug comprises a two-plug system. In some embodiments, the at least one seal is a polymer film that is ultrasonically welded to the porous scaffold.

In some embodiments, the device may comprise a coating, e.g., a biodegradable coating instead of or in addition to a removable plug. In some embodiments, the biodegradable material coats at least a part of the porous scaffold. In some embodiments, the coating is biodegradable and can temporarily isolate the chamber of the implanted device from host tissue. In some embodiments, the material stimulates tissue incorporation and angiogenesis. In some embodiments, the material comprises one or more of a growth factor, an antifibrotic agent, a polymer, vascular endothelial growth factor (VEGF), collagen, fibronectin, polyethylene-imine and dextran sulfate, polyvinyl siloxane and polyethylenimine, phosphorylchloride, poly (ethylene glycol), poly (lactic-co-glycolic acid), poly (lactic acid), polyhydroxyvalerate and copolymers, polyhydroxybutyrate and copolymers, polydiaxanone, polyanhydrides, poly (amino acids), poly (orthoesters), gelatin, a cellulose polymer, a chitosan, an alginate, vinculin, agar, agarose, hyaluronic acid, and Matrigel. In some embodiments, the device further comprises a cell delivery device comprising at least one cell infusion tube configured to be positioned within the chamber and configured to deliver cells to the chamber of the device.

In some embodiments, the device used in the methods described herein comprises:
a porous scaffold comprising an immunologically compatible polymer mesh forming the walls of at least one chamber, wherein the chamber comprises an opening at either or both of a proximal end and a distal end of the chamber, wherein the proximal end and the distal end are separated by a lumen that is bounded by the walls, and wherein the porous scaffold has pores sized to facilitate growth of vascular and connective tissues around and through the walls of the at least one chamber;
at least one removable, non-porous plug configured to be positioned within the lumen of the at least one chamber, wherein the plug extends along the lumen of the chamber; and
at least one seal configured to enclose either or both the proximal end and the distal end of the chamber;

In some embodiments, the device used in the methods described herein comprises: a porous scaffold comprising at least one chamber having a proximal end and a distal end, and at least one removable plug configured to be positioned within the at least one chamber. In some embodiments, the porous scaffold comprises a mesh having pores sized to facilitate growth of vascular and connective tissues into the at least one chamber. In some embodiments, the porous scaffold and/or mesh comprises a polypropylene mesh.

In some embodiments, the device used in the methods described herein comprises: a porous scaffold comprising one or more chambers having a proximal end and a distal end, and an opening at either or both the proximal end and the distal end. In some embodiments, the porous scaffold comprises pores sized to facilitate growth of vascular and connective tissues into the one or more chambers. In some embodiments, the device further comprises one or more two-plug systems comprising an outer plug configured to be positioned within the one or more chambers, and an inner plug configured to be positioned within the outer plug. Additionally, in some embodiments, the device comprises at least one seal configured to enclose the plug system(s) in the chamber and to enclose the opening at either or both the proximal end and the distal end of the chamber.

In some embodiments, the present disclosure provides methods of treating diabetes in a patient in need thereof. An exemplary embodiment is a method of treating diabetes in a patient in need thereof, comprising:
implanting a device in the patient, wherein the device comprises:
   a porous scaffold comprising an immunologically compatible polymer mesh forming the walls of at least one chamber, wherein the chamber comprises an opening at either or both of a proximal end and a distal end of the chamber, wherein the proximal end and the distal end are separated by a lumen that is bounded by the walls, and wherein the porous scaffold has pores sized to facilitate growth of vascular and connective tissues around and through the walls of the at least one chamber;
   at least one removable, non-porous plug configured to be positioned within the lumen of the at least one chamber, wherein the plug extends along the lumen of the chamber; and
   at least one seal configured to enclose either or both the proximal end and the distal end of the chamber;
maintaining the device in the patient's body until the device is infiltrated with vascular and connective tissues;
accessing the implanted device;
withdrawing the plug; and
infusing the chamber with cells, wherein at least some of the cells express insulin, and wherein the insulin-expressing cells are administered at a borderline mass of about 3000 IEQ/kg or greater, preferably suspended in blood, e.g., plasma and/or serum, from the patient.

In some embodiments of the methods disclosed herein (e.g., for treating diabetes), at least about 60%, 65%, 70%, 75%, 80%, 85%, or 90% (e.g., at least 70%) of the cells are purified islets of Langerhans (“islets”). In some embodiments, at least about 70%, 75%, 80%, 85%, or 90% (e.g., at least 80%) of the cells are viable islets. In some embodiments, the cells are purified from a pellet prior to administration. In some embodiments, the pellet has a volume of less than about 15 ml (e.g., less than about 10.5 ml). In some embodiments, the pellet has a volume of less than about 10.5 ml. In some embodiments, the cells are infused in the chamber about 4-24 weeks after implanting the device. In some embodiments, the cells are infused in the chamber about 4, 5, 6, 7, 8, 9, or 10 weeks (e.g., about 6 weeks) after implanting the device. In some embodiments, the cells are infused in the chamber about 6 weeks after implanting the device.

In some embodiments, the methods disclosed herein further comprise administering immunosuppression prior to and/or after infusing the chamber with cells. In some embodiments, the immunosuppression is administered prior to infusion. In some embodiments, the immunosuppression is administered for at least 3, 4, 5, 6, 7, 8, 9, or 10 weeks (e.g., about 6 weeks) prior to infusion. In some embodiments, the immunosuppression is administered for about 6 weeks prior to infusion. In some embodiments, the immunosuppression is administered after infusion. In some embodiments, the immunosuppression is administered for about 7 days after infusion. In some embodiments, the methods disclosed herein further comprise administering etanercept and/or basiliximab.

In some embodiments, the immunosuppression comprises (i) induction therapy prior to cell infusion with thymoglobulin and/or (ii) maintenance therapy comprising one or more of tacrolimus, mycophenolate mofetil, and mycophenolic acid administered after device implantation and/or islet transplantation.

In some embodiments, the immunosuppression comprises induction therapy prior to cell infusion with thymoglobulin. In some embodiments, the administered amount of thymoglobulin comprises a total dose of about 1-10 mg/kg (e.g., about 6 mg/kg) over 1-10 daily infusions (e.g., at least 4 daily infusions). In some embodiments, the administered amount of thymoglobulin comprises a total dose of about 1-10 mg/kg (e.g., about 6 mg/kg). In some embodiments, the administered amount of thymoglobulin comprises a total dose of about 6 mg/kg. In some embodiments, the total dose of thymoglobulin (e.g., about 6 mg/kg) is administered over 1-10 daily infusions (e.g., at least 4 daily infusions). In some embodiments, the total dose of thymoglobulin (e.g., about 6 mg/kg) is administered over at least 4 daily infusions. In some embodiments, the administered amount of thymoglobulin comprises a total dose of about 6 mg/kg over at least 4 daily infusions.

In some embodiments, the immunosuppression comprises maintenance therapy comprising one or more of tacrolimus, mycophenolate mofetil, and mycophenolic acid administered after device implantation and/or islet transplantation. In some embodiments, the administered amount of tacrolimus comprises a dose adjusted upwards daily to a blood level of about 1-10 ng/ml (e.g., about 4-6 ng/ml). In some embodiments, the administered amount of tacrolimus is increased to a blood level of about 7-15 mg/ml (e.g., about 8-10 mg/ml) on the day of cell infusion. In some embodiments, the administered amount of mycophenolate mofetil comprises about 100-750 mg (e.g., 500 mg). In some embodiments, the administered amount of mycophenolate mofetil is increased to about 500-1500 mg (e.g., about 1000 mg) on the day of cell infusion. In some embodiments, the administered amount of mycophenolic acid comprises about 100-500 mg (e.g., about 360 mg). In some embodiments, the administered amount of mycophenolic acid is increased to about 500-1000 mg (e.g., about 720 mg) on the day of cell infusion. In some embodiments, administration of tacrolimus, mycophenolate mofetil, and/or mycophenolic acid commences about 1-5 weeks (e.g., about 3-4 weeks) after device implantation. In some embodiments, administration of tacrolimus, mycophenolate mofetil, and/or mycophenolic acid commences about 3-4 weeks after device implantation.

In some embodiments, the methods disclosed herein further comprise screening a patient for islet function after cell infusion, e.g., by checking for a C-peptide level in a serum sample. In some embodiments, the patient is screened about 1, 2, 3, 4, 5, 6, 7, 8, 9, 10, 11, or 12 months (e.g., about 6 months) after cell infusion by checking for a C-peptide level in a serum sample. In some embodiments, the C-peptide level (e.g., in a serum sample) may be determined one or more times or continuously throughout the period of graft survival in the patient (e.g., about 1 year or more, about 2 years or more, about 5 years or more, about 7 years or more, or about 10 years or more). In some embodiments, the methods disclosed herein further comprise administering a second infusion of cells to a patient having a C-peptide level of less than about 0.2, 0.3, 0.4, or 0.5 ng/ml (e.g., about 0.3 ng/ml). In some embodiments, the methods disclosed herein further comprise administering at least a third or fourth infusion of cells to a patient having a C-peptide level of less than about 0.2, 0.3, 0.4, or 0.5 ng/ml (e.g., about 0.3 ng/ml). In some embodiments, the methods disclosed herein further comprise administering more than four infusions of cells to a patient having a C-peptide level of less than about 0.2, 0.3, 0.4, or 0.5 ng/ml (e.g., about 0.3 ng/ml). In some embodiments, the methods disclosed herein further comprise administering two, three, four, or more infusions of cells to a patient.

In some embodiments, the methods disclosed herein further comprise monitoring a patient for up to one year for insulin-independence after the start of the procedure. In some embodiments, the patient is monitored at about 5, 6, 7, 8, 9, 10, 11, or 12 months after the start of the procedure. In some embodiments, the methods disclosed herein further comprise administering one or more additional infusions of cells to a patient who, at the end of the monitoring period, has a C-peptide level in a serum sample of less than about 0.2, 0.3, 0.4, or 0.5 ng/ml (e.g., about 0.3 ng/ml). In some embodiments, the methods disclosed herein further comprise administering one or more additional infusions of cells to a patient who, at the end of the monitoring period, is not insulin-independent. In some embodiments, the methods disclosed herein further comprise monitoring the patient for up to one year for insulin-independence after the second or subsequent infusion (e.g., after the second, third, or fourth infusion, etc.). In some embodiments, the patient is monitored at about 5, 6, 7, 8, 9, 10, 11, or 12 months after the second or subsequent infusion (e.g., after the second, third, or fourth infusion, etc.). In some embodiments, the methods disclosed herein further comprise monitoring the patient for up to one year for insulin-independence after the third, fourth, or subsequent infusion. In some embodiments, the patient is monitored at about 5, 6, 7, 8, 9, 10, 11, or 12 months after the third, fourth, or subsequent infusion. In some embodiments, the methods disclosed herein further comprise monitoring the patient one or more times or continuously throughout the period of graft survival. In some embodiments, the period of graft survival may be about 1, 2, 3, 4, 5, 6, 7, 8, 9, or 10 years or more (e.g., about 1 year or more, about 2 years or more, about 5 years or more, about 7 years or more, or about 10 years or more).

In some embodiments of the methods disclosed herein (e.g., for treating diabetes), the patient has type 1 diabetes mellitus (T1DM) with hypoglycemia unawareness. In some embodiments, the patient has type one diabetes mellitus with hypoglycemia unawareness as measured by, e.g., i) a Clarke reduced awareness score of 3, 4, 5, or more (e.g., about 4 or more); ii) a HYPO score greater than or equal to the 90th percentile (e.g., about 1047) during the screening period and within the last 6 months; iii) one or more swings in blood glucose despite diabetes therapy, as defined by an LI score greater than or equal to the 90th percentile (e.g., about 433 mmol/L²/h wk⁻¹) during the screening period and/or within the 6 months prior to treatment; and/or iv) a composite Clarke score of about 4 or more and a HYPO score greater than or equal to about the 75th percentile and a LI greater than or equal to about the 75th percentile (e.g., about 329) during the screening period and/or within the 6 months prior to treatment. In some embodiments, the patient has a history of severe hypoglycemic episodes. In some embodiments, the patient has required insulin for about 2, 3, 4, 5, 6, 7, 8, 9 or 10 or more years (e.g., at least about 5 years). In some embodiments, the patient is between about 18 and 65 years of age. In some embodiments, the patient has less than about 0.3 ng/ml C-peptide in a serum sample prior to treatment in response to a mixed meal tolerance test (e.g., a meal test using Boost® 6 mL/kg body weight to a maximum of 360 mL). In some embodiments, the patient has less than about 0.3 ng/ml C-peptide in a serum sample prior to treatment in response to a mixed meal tolerance test (e.g., a meal test using Boost® 6 mL/kg body weight to a maximum of 360 mL) as measured during an about 1, 2, 3, 4, or 5 hour test (e.g., an about 2 hour test). In some embodiments, the patient is restored to normoglycemia.

In some embodiments, the methods disclosed herein comprise implanting 1, 2, 3, 4, 5, 6, 7, 8, 9, 10 devices (e.g., 2-6 devices, e.g., 2-4 devices) in the patient. In some embodiments, the methods disclosed herein comprise implanting 2-6 devices (e.g., 2-4 devices) in the patient. In some embodiments, the device or devices are implanted in the patient's abdomen. In some embodiments, the methods disclosed herein further comprise administering the patient Cephazolin and/or Keflex. In some embodiments, the methods disclosed herein further comprise a step of imaging the porous scaffold prior to delivering cells.

In some embodiments of the methods disclosed herein (e.g., for treating diabetes), the cells comprise genetically engineered cells that express insulin. In some embodiments, the cells comprise islets and one or more of Sertoli cells, mesenchymal stem cells, differentiated stem cells, and genetically engineered cells. In some embodiments, the cells comprise stem cells or stem cell-derived cells. In some embodiments, the cells comprise allogeneic, xenogeneic, or syngeneic donor cells, or patient-derived cells. In some embodiments, the cells comprise genetically engineered cells or cell lines. In some embodiments, the cells comprise encapsulated cells. In some embodiments, the cells are encapsulated in alginate, a polysaccharide hydrogel, chitosan, calcium or barium alginate, a layered matrix of alginate and polylysine, photopolymerizable poly (ethylene glycol) polymer, a polyacrylate, hydrogel methacrylate, methyl methacrylate, a silicon capsule, a silicon nanocapsule, a polymembrane, or acrylonitrile-co-vinyl chloride. In some embodiments, the cells comprise two or more cell types selected from islets, Sertoli cells, stem cells, differentiated stem cells, embryonic stem cells, induced pluripotent stem cells (iPSC), allogeneic cells, xenogeneic or syngeneic cells, and genetically engineered cells or cell lines.

### BRIEF DESCRIPTION OF THE DRAWINGS

FIGS. 1A-1E illustrate various embodiments of an exemplary single-chamber device consistent with the present disclosure.

FIG. 1F illustrates an embodiment of an exemplary multi-chamber device consistent with the present disclosure.

FIGS. 2A-2D illustrate various mesh configurations that may be used in forming an exemplary device consistent with the present disclosure.

FIG. 3A illustrates an embodiment of an exemplary device consistent with the present disclosure. FIG. 3B illustrates components of the exemplary device shown in FIG. 3A.

FIG. 4 illustrates a porous scaffold of an exemplary device consistent with the present disclosure.

FIG. 5A illustrates a seal of an exemplary device consistent with the present disclosure. FIG. 5B is a cross-sectional view of the seal shown in FIG. 5A.

FIG. 6A illustrates multiple outer plugs of a two-part plug system of an exemplary device consistent with the present disclosure. FIG. 6B is a cross-sectional view of one of the outer plugs shown in FIG. 6A. FIG. 6C is a cross-sectional view of a plug system and a single porous scaffold assembly prior to implantation in a host body. FIG. 6D is a cross-sectional view of the assembly shown in FIG. 6C following incubation in a host body. FIG. 6E is a cross-sectional view of a porous scaffold implanted in a host body following removal of the plug system.

FIG. 7 illustrates multiple inner plugs of a two-part plug system of an exemplary device consistent with the present disclosure.

FIG. 8 illustrates a seal for enclosing cells within a vascularized chamber of an exemplary device consistent with the present disclosure.

FIG. 9A illustrates a device for delivering cells to an exemplary device consistent with the present disclosure. FIG. 9B illustrates a cell infusion mechanism of the delivery device shown in FIG. 9A. FIG. 9C illustrates additional steps of the cell infusion mechanism of the delivery device shown in FIGS. 9A-9B.

FIG. 10 illustrates a flow chart showing steps of an exemplary cell transplantation method in accordance with the present disclosure.

FIGS. 11A-11D illustrate a schematic overview of certain steps of an exemplary cell infusion procedure in accordance with the present disclosure.

FIG. 12 illustrates a schematic diagram of an exemplary device in accordance with the present disclosure.

FIG. 13A-13B illustrate Continuous Glucose Monitoring (CGM) in an exemplary treated patient. Shaded areas indicate a blood glucose level range of 70-180 mg/dL. FIG. 13A illustrates blood glucose levels over time (mg/dL) at baseline. FIG. 13B illustrates blood glucose levels over time (mg/dL) at 90 days post-transplant.

FIG. 14 illustrates a representative histological image from the islet-transplanted mini CP-device explanted from an exemplary treated patient at 90 days post-transplant. Islets stained for insulin are shown in red and new blood vessels stained for von Willebrand Factor (vWF) are shown green.

FIG. 15 illustrates a representative histological image of donor islets transplanted into a CP-device in an exemplary treated patient (5 micron serial sections, paraffin embedded). Immunofluorescence staining for insulin (INS) is shown in red and for cell nuclei (DAPI) in blue. A TBS (Tris-buffered saline) only (no primary antibody) negative control image is also shown.

FIG. 16A-16B illustrate representative Masson's Trichrome (MT) (FIG. 16A) and Hematoxylin and Eosin (H&E) (FIG. 16B) scans of a segment (AQ0160 20) from the mini CP-device removed from an exemplary treated patient (5 micron serial sections, paraffin embedded). FIG. 16C illustrates positive immunofluorescence staining (AQ0160 21) for Insulin (INS, red) and von Willebrand Factor (vWF, green) and cell nuclei (DAPI, blue). A TBS only (no primary antibody) negative control image is also shown.

FIG. 17A-17B illustrate representative immunofluorescence images of a segment (AQ0160 23/24) from the mini CP-device removed from an exemplary treated patient (5 micron serial sections, paraffin embedded). FIG. 17A illustrates positive immunofluorescence staining (AQ0160 23) for Insulin (INS) and Glucagon (GLUC) [Red: Insulin, Green: Glucagon, Blue: DAPI (Nuclei)]. A TBS only (no primary antibody) negative control image is also shown. FIG. 17B illustrates positive immunofluorescence staining (AQ0160 24) for Insulin (INS) and Somatostatin (SOM) [Red: Insulin, Green: Somatostatin, Blue: DAPI (Nuclei)]. A TBS only (no primary antibody) negative control image is also shown.

FIG. 18A-18B illustrate representative immunofluorescence images of a segment (AQ0160 25/26) from the mini CP-device removed from an exemplary treated patient (5 micron serial sections, paraffin embedded). FIG. 18A illustrates positive immunofluorescence staining (AQ0160 25) for Insulin (INS) [Red: Insulin, Blue: DAPI (Nuclei)]. A TBS only (no primary antibody) negative control image is also shown. FIG. 18B illustrates positive immunofluorescence staining (AQ0160 26) for C-peptide [Green: C-peptide, Blue: DAPI (Nuclei)]. A TBS only (no primary antibody) negative control image is also shown.

FIG. 19 illustrates a representative immunofluorescence image of a segment (AQ0160 27) from the mini CP-device removed from an exemplary treated patient (5 micron serial sections, paraffin embedded). Positive immunofluorescence staining (AQ0160 27) for Insulin (INS) and CK 19 is shown [Red: Insulin, Green: CK 19, Blue: DAPI (Nuclei)]. A TBS only (no primary antibody) negative control image is also shown.

### DETAILED DESCRIPTION OF EXEMPLARY EMBODIMENTS

The disclosed methods and compositions may be understood more readily by reference to the following detailed description taken in connection with the accompanying figures, which form a part of this disclosure.

Throughout this text, the descriptions refer to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

When a range of values is expressed, the range includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. As used herein, the singular forms “a,” “an,” and “the” include plural forms unless the context clearly dictates otherwise. The use of “or” will mean “and/or” unless the context clearly dictates otherwise. All references cited herein are incorporated by reference for any purpose. Where a reference and the specification conflict, the specification will control.

It is to be appreciated that certain features of the disclosed methods and compositions, which are, for clarity, described herein in the context of separate embodiments, may also be provided in combination in a single embodiment. Conversely, various features of the disclosed methods and compositions that are, for brevity, described in the context of a single embodiment, may also be provided separately or in any sub-combination.

Various terms relating to aspects of the description are used throughout the specification and claims. Such terms are to be given their ordinary meaning in the art unless otherwise indicated. Other specifically defined terms are to be construed in a manner consistent with the definitions provided herein. Throughout the disclosure, the terms cell infusion and cell transplantation are used interchangeably.

In certain aspects, the present disclosure provides methods of treating a disease, disorder, or condition (e.g., diabetes) in a patient in need thereof. In certain aspects, the present disclosure provides methods of treating diabetes in a patient in need thereof. In some embodiments, the methods described herein (e.g., for treating diabetes) comprise: implanting a device in a patient; maintaining the device in the patient's body until the device is infiltrated with vascular and connective tissues; accessing the implanted device; withdrawing a plug from the device; and infusing the chamber with cells. In some embodiments, at least some of the cells express insulin. In some embodiments, the cells and/or the insulin-expressing cells are administered at a borderline mass of about 3000 IEQ/kg or greater. In some embodiments, the cells and/or the insulin-expressing cells are suspended in blood (e.g., plasma and/or serum) from the patient. In some embodiments, the cells and/or the insulin-expressing cells are suspended in plasma from the patient. In some embodiments, the cells and/or the insulin-expressing cells are suspended in plasma from the patient, wherein the suspension of cells in plasma further comprises one or more growth factors. In some embodiments, the cells and/or the insulin-expressing cells are suspended in serum from the patient. In some embodiments, the cells and/or the insulin-expressing cells are suspended in serum from the patient, wherein the suspension of cells in serum further comprises one or more growth factors. In some embodiments, the blood, plasma, and/or serum contains one or more growth factors. In some embodiments, the blood, plasma, and/or serum (e.g., with or without one or more growth factors) contains one or more proteins that may provide nutrients to the cells, signal for improved vascularization and/or regeneration, and/or alleviate inflammation.

In some embodiments, the methods disclosed herein use at least one device for delivering, containing, and/or maintaining cells (e.g., therapeutic cells, e.g., insulin-expressing and/or islets) in vivo.

In some embodiments, the device comprises: a porous scaffold comprising an immunologically compatible polymer mesh forming the walls of at least one chamber; at least one plug configured to be positioned within the lumen of the at least one chamber; and at least one seal configured to enclose either or both of a proximal end and a distal end of the at least one chamber. In some embodiments, the porous scaffold comprises an immunologically compatible polymer mesh. In some embodiments, the at least one plug comprises at least one removable, non-porous plug.

More specifically, in some embodiments, the device comprises: a porous scaffold comprising an immunologically compatible polymer mesh forming the walls of at least one chamber, wherein the chamber comprises an opening at either or both of a proximal end and a distal end of the chamber, wherein the proximal end and the distal end are separated by a lumen that is bounded by the walls, and wherein the porous scaffold has pores sized to facilitate growth of vascular and connective tissues around and through the walls of the at least one chamber; at least one removable, non-porous plug configured to be positioned within the lumen of the at least one chamber, wherein the plug extends along the lumen of the chamber; and at least one seal configured to enclose either or both the proximal end and the distal end of the chamber. In some embodiments, the device further comprises a cell delivery device comprising at least one cell infusion tube configured to be positioned within the chamber and configured to deliver cells to the chamber of the device.

In some embodiments, the device comprises: at least one porous scaffold comprising a chamber therein and having an opening at either or both a proximal end and a distal end of the scaffold; and at least one plug configured to be housed in the chamber. In some embodiments, the opening at one or both the ends of the chamber is sized to enable insertion and/or retraction of the plug from the chamber. In some embodiments, the at least one porous scaffold is tubular in shape, and/or the at least one plug is cylindrical and extends along a lumen of the at least one porous scaffold. In some embodiments, the porous scaffold is open only at the proximal end. In some embodiments, the distal end of the tubular porous scaffold comprises a rounded or flat-bottomed surface. In some embodiments, the edges at the distal end of the porous scaffold are tapered and/or brought into contact with one another to seal the distal end.

In some embodiments, the device comprises: a porous scaffold comprising one or more chambers having a proximal end and a distal end. In some embodiments, the one or more chambers comprise an opening at the proximal end. In some embodiments, the device further comprises one or more plug systems comprising an outer plug configured to be positioned within the one or more chambers, and an inner plug configured to be positioned within the outer plug. Additionally, in some embodiments, the device comprises at least one seal configured to enclose the plug system within the chamber and/or seal the opening at the proximal end of the chamber. In some embodiments, the porous scaffold of the device used in the methods described herein is formed of a biocompatible material that elicits a mild inflammatory response in the body. In some embodiments, the mild inflammatory response stimulates angiogenesis and/or promotes incorporation of a vascularized collagen matrix into the device, but does not result in significant inflammation around the device. An example of such a biocompatible material is polypropylene. In some embodiments, the porous scaffold comprises a woven polypropylene mesh that has sufficient stiffness to facilitate device fabrication. In some embodiments, the woven polypropylene mesh is selected to allow microvessels to enter the device and/or be maintained as robust, healthy vessels. In some embodiments, such function(s) can be critical for the survival and/or normal functioning of the therapeutic cells infused into the device.

By encouraging regulated growth of vascularized tissue into the device, in some embodiments, the porous scaffold prevents encapsulation of the device by scar tissue. In some embodiments, ingrown tissues stabilize the implant and/or prevent inadvertent movement of the device in situ. Additionally, in some embodiments, at least part of the porous scaffold is coated with a material (e.g., one or more biological or non-biological agents). In some embodiments, the material stimulates tissue incorporation and/or angiogenesis. In some embodiments, the material comprises one or more of a growth factor, an antifibrotic agent, a polymer, vascular endothelial growth factor (VEGF), collagen, fibronectin, polyethylene-imine and dextran sulfate, polyvinyl siloxane and polyethylenimine, phosphorylchloride, poly (ethylene glycol), poly (lactic-co-glycolic acid), poly (lactic acid), polyhydroxyvalerate and copolymers, polyhydroxybutyrate and copolymers, polydiaxanone, polyanhydrides, poly (amino acids), poly (orthoesters), gelatin, a cellulose polymer, a chitosan, an alginate, vinculin, agar, agarose, hyaluronic acid, and Matrigel. For example, the device and/or porous scaffold may be dip-coated in a polymer-drug formulation, or another technique may be used to apply the material to the device and/or scaffold. Examples of biological or non-biological agents capable of stimulating tissue incorporation and/or angiogenesis include but are not limited to: VEGF (vascular endothelial growth factor), PDGF (platelet-derived growth factor), FGF-1 (fibroblast growth factor), NRP-1 (neuropilin-1), Ang-1, Ang-2 (angiopoietin 1, 2), TGF-β, endoglin, MCP-1, αvβ3, αvβ5, CD-31, VE-cadherin, ephrin, plasminogen activators, angiogenin, Del-1, aFGF (acid fibroblast growth factor), vFGF (basic fibroblast growth factor), follistatin, G-CSF (granulocyte colony-stimulating factor), HGF (hepatocyte growth factor), II-8 (interleukin-8), Leptin, midkine, placental growth factor, PD-ECGF (platelet-derived endothelial growth factor), PTN (pleiotrophin), progranulin, proliferin, TGF-α, and TNF-α.

In some embodiments, the outer surface of the porous scaffold is roughened, e.g., to stimulate tissue ingress. In some embodiments, the porous scaffold includes various drug-eluting polymer coatings. In some embodiments, the porous scaffold may be coated with a biodegradable or non-biodegradable polymer without a drug. In some embodiments, the porous scaffold may be partially or completely coated with the polymer. Representative polymers that can be used for coating and/or drug elution include but are not limited to: methacrylate polymers, polyethylene-imine and dextran sulfate, poly (vinylsiloxane) ecopolymere-polyethyleneimine, phosphorylcholine, poly (ethyl methacrylate), polyurethane, poly (ethylene glycol), poly (lactic-glycolic acid), hydroxyapetite, poly (lactic acid), polyhydroxyvalerte and copolymers, polyhydroxybutyrate and copolymers, polycaprolactone, polydiaxanone, polyanhydrides, polycyanocrylates, poly (amino acids), poly (orthoesters), polyesters, collagen, gelatin, cellulose polymers, chitosans, and alginates or combinations thereof. Additional examples of polymers that may be used to coat at least a part of the porous scaffold include but are not limited to: collagen, fibronectin, extracellular matrix proteins, vinculin, agar, and agarose. Various mixtures of polymers may also be used.

With respect to drug elution, in some embodiments, at least a part of the porous scaffold may include an antibiotic coating, e.g., to minimize infections. Representative antibiotics include but are not limited to: ampicillin, tetracycline, nafcillin, oxacillin, cloxacillin, dicloxacillin, flucloxacillin, vancomycin, kanamycin, gentamicin, streptomycin, clindamycin, trimethoprim/sulfamethoxazole, linezolid, teicoplanin, erythromycin, ciprofloxacin, rifampin, penicillin, amoxicillin, sulfonamides, nalidixic acid, norfloxacin, ciprofloxacin, ofloxacin, sparfloxacin, lomefloxacin, fleroxacin, pefloxacin, amifloxacin, 5-fluorouracil, chloramphenicol, polymyxin, mitomycin, chloroquin, novobiocin, nitroimadazole. In some embodiments, the porous scaffold includes a bactericidal agent. Representative bactericidal agents include but are not limited to: benzalkonium chloride, chlorohexidine gluconate, sorbic acid and salt thereof, thimerosal, chlorobutanol, phenethyl alcohol, and p-hydroxybenzoate.

In some embodiments, at least a part of the device and/or porous scaffold may be coated with one or more antifibrotic agents, e.g., to inhibit fibrous tissue encapsulation. Representative antifibrotic agents include but are not limited to: paclitaxel, everolimus, tacrolimus, rapamycin, halofuginone hydrobromide, combretastatin and analogues and derivatives thereof (such as combretastatin A-1, A-2, A-3, A-4, A-5, A-6, B-1, B-2, B-3, B-4, D-1, D-2, and combretastatin A-4 phosphate), docetaxel, vinblastine, vincristine, vincristine sulfate, vindesine, and vinorelbine, camptothecin topotecan, irinotecan, etoposide or teniposide anthramycin, mitoxantrone, menogaril, nogalamycin, aclacinomycin A, olivomycin A, chromomycin A3, and plicamycin, methotrexate, edatrexate, trimetrexate, raltitrexed, piritrexim, denopterin, tomudex, pteropterin, and derivatives and analogues thereof. In some embodiments, the device and/or porous scaffold may also include polymethyl methacrylate or bone cement or other types of cyanoacrylates.

In some embodiments, the porous scaffold is formed of a material that allows imaging of the implanted device using, e.g., MRIs, fMRIs, CT scans, X-rays, ultrasounds, PET scans, etc. In some embodiments, the porous scaffold comprises a polymer mesh (e.g., polypropylene, polytetrafluoroethylene (PTFE), polyurethane, polyesters, silk meshes, etc.) that is immunologically compatible and/or allows imaging of the neovascularized tissue. In some embodiments, the porous scaffold comprises a combination of materials. In some embodiments, the porous scaffold comprises interwoven polypropylene and silk strands.

In some embodiments, the pore size of the scaffold material is selected to facilitate tissue incorporation and/or vascularization within at least one chamber of the porous scaffold. In some embodiments, the pore size of the scaffold material is selected to facilitate growth of vascular and connective tissues around and through the walls of at least one chamber of the porous scaffold. In some embodiments, the pore sizes may range from about 50 nm to 5 mm. In some embodiments, the porous scaffold comprises a woven polypropylene mesh with a 0.53 mm pore diameter.

In some embodiments, the pore size of the scaffold material is selected to exclude immune cells or immune agents from penetrating the implanted device. In some embodiments, the pore size does not necessarily need to exclude immune cells or immune agents from infiltrating the device. This may be the case, for example, when the device is used to transplant a combination of cells, including immunoprotective cells, (e.g., Sertoli cells, mesenchymal stem cells, etc.) that can provide immune protection to the co-transplanted cells. This may also be the case, for example, when the device is used to transplant syngeneic cells, or cells derived from the patient receiving the transplant.

In some embodiments, the porous scaffold comprises at least one chamber. In some embodiments, the porous scaffold comprises one chamber, two chambers, three chambers, four chambers, five chambers, six chambers, seven chambers, eight chambers, ten chambers, twelve chambers, or more chambers. In some embodiments, the porous scaffold comprises about eight chambers, about nine chambers, or about ten chambers. In some embodiments, the porous scaffold comprises multiple chambers that are connected laterally. In some embodiments, the at least one chamber comprises an opening at either or both of a proximal end and a distal end of the chamber. In some embodiments, the proximal end and the distal end of the chamber are separated by a lumen that is bounded by the walls. In some embodiments, the porous scaffold is one of the exemplary scaffolds disclosed in Intl. Application No. PCT/US2010/047028 (Intl. Publication No. WO 2011/025977), which is incorporated herein by reference for disclosure of exemplary scaffolds, plugs and plug systems, seals, and cell transplantation and/or delivery devices and methods.

In some embodiments, the plug or plug system of the device used in the methods described herein is configured to fit into the chamber within the porous scaffold. In some embodiments, the plug or plug system may comprise a non-porous material (e.g., polytetrafluoroethylene (PTFE), polypropylene, etc.), e.g., to inhibit ingrowth of biological tissue into the plug or plug system. The plug or plug system may be a hollow or solid structure. However, if a hollow plug is used, care should be taken to prevent infiltration of collagen or any other biological material into the lumen of the plug when the device is implanted into host tissue. In some embodiments, at least one removable, non-porous plug extends along the lumen of the chamber within the porous scaffold. In some embodiments, the at least one removable, non-porous plug comprises a two-plug system. In some embodiments, the plug or plug system is one of the exemplary plugs or plug systems disclosed in Intl. Application No. PCT/US2010/047028 (Intl. Publication No. WO 2011/025977), which is incorporated herein by reference for disclosure of exemplary scaffolds, plugs and plug systems, seals, and cell transplantation and/or delivery devices and methods.

In some embodiments, the proximal end of the plug or plug system is connected to at least one seal. In some embodiments, the seal is configured to close the proximal opening of the chamber when the plug or plug system is completely inserted into the chamber of the porous scaffold. In some embodiments, the seal is structured to hold the plug or plug system in place inside the porous scaffold. In some embodiments, the seal is separate from the plug or plug system. In some embodiments, the seal is connected to the porous scaffold. In some embodiments, the seal is a polymer film that is ultrasonically welded to the porous scaffold. In some embodiments, the proximal end of the chamber is closed using surgical sutures and/or vascular clips without using a separate seal. In some embodiments, the seal is one of the exemplary seals disclosed in Intl. Application No. PCT/US2010/047028 (Intl. Publication No. WO 2011/025977), which is incorporated herein by reference for disclosure of exemplary scaffolds, plugs and plug systems, seals, and cell transplantation and/or delivery devices and methods.

When implanted in a host body, in some embodiments, the porous scaffold of the device used in the methods described herein encourages ingrowth of vascular and/or connective tissue, such that the plug or plug system housed within the scaffold becomes encapsulated in a vascularized tissue matrix. In some embodiments, when the plug or plug system is removed from the porous scaffold, a neovascularized chamber is created within the device. In some embodiments, the neovascularized chamber can then be used for holding a cell preparation in the host body.

In some embodiments, the sizes of the porous scaffold and/or the plug or plug system are selected to provide an optimal surface area-to-volume ratio for holding cells in vivo and/or for ensuring long-term survival of the cells within the neovascularized chamber. Similarly, in some embodiments, the number of chambers in the device may be determined based on the volume and/or number of cells that are to be transplanted. In some embodiments, the total volume of the cell device is adjusted by increasing or decreasing the number of chambers while maintaining an optimum surface area-to-volume ratio of each individual chamber. In some embodiments, the length of the chambers is adjusted to alter the total volume. Alternatively, in some embodiments, the device comprises a fixed number of chambers, but only a selected number of chambers are infused with cells depending on the total volume requirement of the device. In some embodiments, the length of the chambers and/or the number of chambers is adjusted to alter the total volume required.

In some embodiments, the device used in the methods described herein is implanted in a host body, e.g., a patient's body, e.g., a diabetic patient's body. In some embodiments, the device can be implanted either subcutaneously or intraperitoneally in a host body, including the omentum or other appropriate site. Alternatively, in some embodiments, the device can be implanted partially intraperitoneally in a host body, including into the omentum or other appropriate site, and extend into the subcutaneous environment. In some embodiments, cells may be loaded in the portion of the device extending into the subcutaneous environment while the rest of the device is in the intraperitoneal environment. In some embodiments, the device may be implanted into the brain, spinal cord area, or any other organ as required to elicit a therapeutic effect from the transplanted cells. In some embodiments, the device is implanted in a position to allow the transplanted cells to remain equally or nearly equally dispersed within the chambers of the device. For instance, in some embodiments, the device may be implanted subcutaneously in a host body such that the chambers of the device are parallel to the host's waist line. In some embodiments, the device is implanted in a position to limit the potential risk of infection and/or fluid collection (e.g., seroma). For instance, in some embodiments, the device may be implanted subcutaneously in the sublay (retromuscular) position. In some embodiments, the device is implanted subcutaneously just under the superficial abdominal fascia. In some embodiments, the host is a human. In some embodiments, the host is another mammalian or non-mammalian animal.

In some embodiments, the cell transplantation procedure is a two-step process comprising a device implantation step followed by a cell infusion (cell transplantation) step. In some embodiments, the cell infusion step is implemented after an in vivo incubation period during which the implanted device is infiltrated with a vascularized collagen matrix. In some embodiments, the incubation period is about 1 to about 60, about 5 to about 50, about 10 to about 40, about 15 to about 35, or about 20 to about 30 days. In some embodiments, the incubation period is approximately 30 days. In some embodiments, the incubation period is approximately 40 days. In some embodiments, the incubation period is about 1 to about 24, about 4 to about 24, about 1 to about 10, about 4 to about 10, about 2 to about 6, about 2 to about 8, about 3 to about 6, about 3 to about 5, about 3 to about 4, or about 4 to about 6 weeks. In some embodiments, the incubation period is approximately 2 to 8 weeks. In some embodiments, the incubation period is approximately 3 to 6 weeks. In some embodiments, the incubation period is approximately 4 to 6 weeks. In some embodiments, the incubation period is approximately 3 weeks. In some embodiments, the incubation period is approximately 4 weeks. In some embodiments, the incubation period is approximately 5 weeks. In some embodiments, the incubation period is approximately 6 weeks. In some embodiments, the incubation period allows adequate time for angiogenesis and/or collagen infiltration of the porous scaffold. The incubation period may be lengthened or shortened, depending on the degree of neovascularization and tissue (collagen with cells) formation needed or desired. For example, in some embodiments, devices may vascularize at different rates depending on the device material, dimensions, or coatings, such as, e.g., antibiotic coatings, growth factors, etc. Devices may also vascularize at different rates in different hosts, and/or when located in different body tissues within the same host. It is within the skill of a person in the art to determine the appropriate incubation period. For example, imaging studies may be performed prior to delivering cells to ensure that adequate vascular and/or connective tissue is deposited around and through the walls of the porous scaffold during the incubation period.

For the cell infusion step, in some embodiments, the implantation site is accessed through a surgical incision, and the plug or plug system is removed from the porous scaffold to create a collagen and blood vessel lined pocket within the scaffold. In some embodiments, the cell preparation is then delivered into the vascularized pocket, and the porous scaffold is re-sealed. In some embodiments, the cell transplantation procedure is a single step process wherein the device is placed and the cells implanted at the same time. In some embodiments, the cells may be placed in a matrix, e.g., to prevent the cells from leaking through the pores of the device. In some embodiments, the device may be coated with a degradable polymer, e.g., to prevent cells from leaking from the device during the process of angiogenesis and/or collagen development.

In some embodiments, the cells to be transplanted may be combined with a biocompatible viscous solution or biodegradable polymer formulation prior to being loaded into the chamber of the device used in the methods described herein. In some embodiments, the biodegradable polymer formulation protects the cells until the device is fully vascularized by the host body. In some embodiments, such formulations may be placed into the chamber prior to or following placement of the device in a host, but before a collagen matrix and/or vascular structures have formed in the device. In some embodiments, cells combined with a biocompatible viscous solution or biodegradable polymer formulation may be particularly useful in devices designed to be loaded with cells prior to implantation of the device in the host body. Representative polymers that can be used as a biodegradable formulation with cells include but are not limited to: polyethyleneimine and dextran sulfate, poly(vinylsiloxane) ecopolymerepoly-ethyleneimine, phosphorylcholine, poly(ethylene glycol), poly(lactic-glycolic acid), poly(lactic acid), polyhydroxyvalerte and copolymers, polyhydroxybutyrate and copolymers, polydiaxanone, polyanhydrides, poly (amino acids), poly (orthoesters), polyesters, collagen, gelatin, cellulose polymers, chitosans, alginates, fibronectin, extracellular matrix proteins, vinculin, agar, agarose, hyaluronic acid, Matrigel, and combinations thereof.

In some embodiments, the cells to be transplanted may be re-suspended in a patient's own blood (e.g., plasma and/or serum). In some embodiments, the blood (e.g., plasma and/or serum) replaces transplant media. In some embodiments, the blood, plasma, and/or serum contains one or more growth factors. In some embodiments, the blood, plasma, and/or serum contains one or more proteins that may provide nutrients to the cells, signal for improved vascularization and/or regeneration, and/or alleviate inflammation.

In some embodiments, the cells placed in the device may also be encapsulated. Non-limiting examples of polymeric cell encapsulation systems include alginate encapsulating, polysaccharide hydrogels, chitosan, calcium or barium alginate, a layered matrix of altinate and polylysine, photopolymerizable poly (ethylene glycol) polymer to encapsulate individual cells or cell clusters, polyacrylates including hydroxyethyl methacrylate methyl methacrylate, silicon capsules, silicon nanocapsules, and polymembrane (acrylonitrile-co-vinyl chloride).

In some embodiments, the device used in the methods described herein is one of the exemplary devices disclosed in Intl. Application No. PCT/US2010/047028 (Intl. Publication No. WO 2011/025977), which is incorporated herein by reference. In some embodiments, the device used in the methods described herein is one of the exemplary devices discussed herein and/or illustrated in one or more of the accompanying figures.

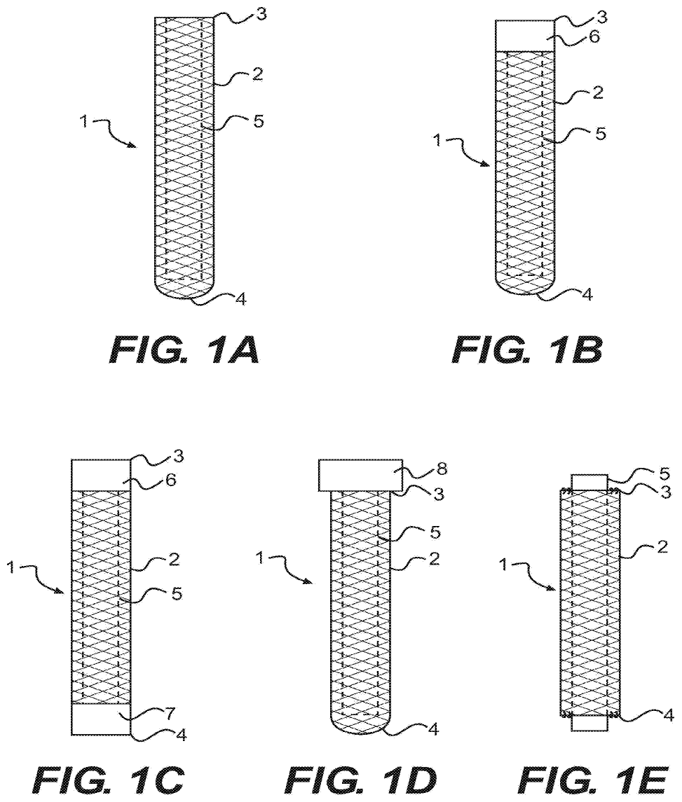

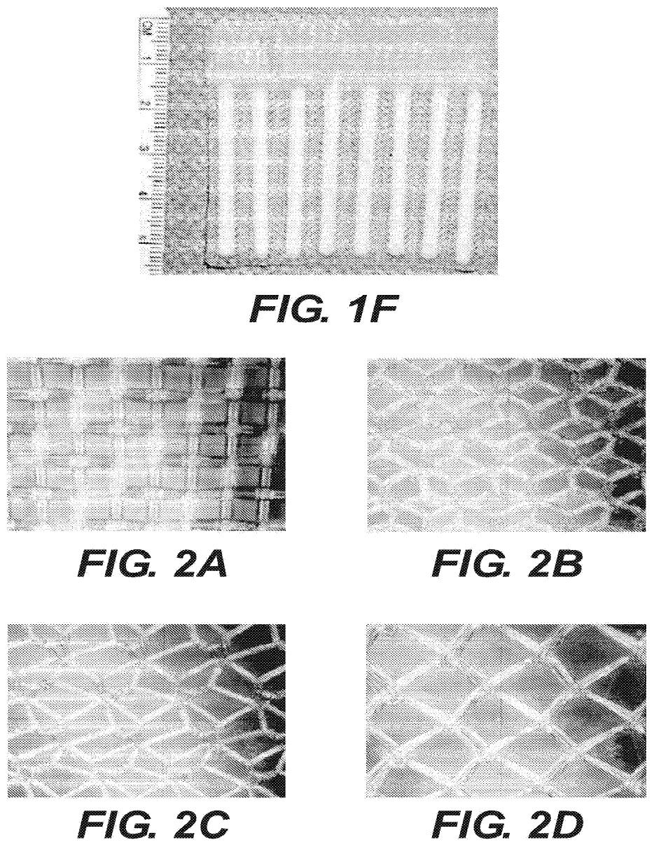

FIGS. 1A-1E show various exemplary embodiments of an exemplary device 1. Device 1 comprises a polymer mesh (e.g., a polypropylene mesh, a PTFE mesh, or any other suitable material) that forms a porous chamber 2 for containing cells in a host body. In some embodiments, device 1 may comprise 1, 2, 3, 4, 5, 6, 7, 8, 9, 10, 11, 12 or more porous chambers 2. In some embodiments, the availability of multiple chambers allows the use of any number or combination of chambers depending on the volume of cellular preparation required, which is within the knowledge and skill of persons skilled in the art to determine.

As shown in FIG. 1A, device 1 comprises a proximal end 3, a distal end 4, and a plug 5 housed in porous chamber 2. In some embodiments, porous chamber 2 is tubular in shape, and plug 5 is cylindrical and extends along a lumen of porous chamber 2. In some embodiments, porous chamber 2 comprises an opening at proximal end 3. In some embodiments, the opening at proximal end 3 is sized to enable insertion and/or retraction of plug 5 from porous chamber 2. In some embodiments, the opening at proximal end 3 is sealed using surgical sutures and/or vascular clamps during device incubation and after infusion of cells into the device. As would be understood by a person of ordinary skill in the art, any other surgical sealing element, e.g., microvascular clips, clamps, etc., can be used to seal the opening at proximal end 3. In some embodiments, device 1 comprises a non-porous flap 6 at proximal end 3, as shown in FIG. 1B. In some embodiments, flap 6 is made of silicone. Flap 6, in some embodiments, can be sealed using surgical sutures, clamps, or any other suitable sealing mechanism during device incubation and after infusion of cells into the device. In some embodiments, distal end 4 of device 1 comprises a rounded or flat-bottomed surface. In some embodiments, device 1 comprises an opening at distal end 4, which can be sealed using surgical sutures, clamps, or any other surgical sealing element, during device incubation and after infusion of cells. In some embodiments, as shown in FIG. 1C, distal end 4 comprises a non-porous portion 7, which may prevent tissue ingrowth at the distal end of the device and/or facilitate retraction of plug 5 from the device prior to cell infusion.

In some embodiments, as shown in FIG. 1D, the proximal end of plug 5 is connected to a seal 8. In some embodiments, seal 8 is configured to close the opening at proximal end 3 when plug 5 is inserted into chamber 5. In some embodiments, seal 8 is structured to hold plug 5 in place inside the porous chamber. In some embodiments, plug 5 is longer than porous chamber 2 and acts as a seal on both proximal end 3 and distal end 4 of the device, as shown in FIG. 1E. In some embodiments, the edges of porous chamber 2 around plug 5 are sealed using surgical sutures and/or surgical glue. After removal of plug 5 prior to cell infusion, in some embodiments, the openings at proximal end 3 and distal end 4 can be sealed using surgical sutures, vascular clamps, or any other suitable sealing mechanism, as would be understood by one of ordinary skill in the art.

In some embodiments, device 1 comprises multiple porous chambers 2 that are laterally connected to each other. In some embodiments, the multiple porous chambers 2 are formed, e.g., by ultrasonically welding the top and bottom surfaces of a porous material along a line substantially parallel to a longitudinal axis of the device. FIG. 1F shows an exemplary device having eight porous chambers 2. Each chamber 2 can house a plug 5 during the device incubation phase. In some embodiments, plugs 5 are removed from chambers 2 prior to infusion of cells into the chambers. In some embodiments, device 1 comprises eight porous chambers and has an overall length of 50 mm and width of 45 mm. Each porous chamber 2 has an inner diameter no greater than 3.5 mm and houses a plug 5 having a length of approximately 40 mm and diameter 2.5 mm. In some embodiments, plug 5 is formed of a non-porous, biocompatible material, e.g., polytetrafluoroethylene (PTFE).

In some embodiments, exemplary device(s) used in the methods of the present disclosure are formed of medical grade polypropylene meshes, e.g., Polypropylene Knitted Mesh (PPKM) (SURGICALMESH™, Brookfield, Connecticut, USA). In some embodiments, the meshes are formed of monofilaments ranging in diameter from 0.1 mm to 0.3 mm, and/or mesh pore sizes ranging from 0.3 mm to 1 mm, from 0.4 mm to 0.85 mm, and 0.5 mm to 0.6 mm. FIGS. 2A-2D show various exemplary mesh configurations that may be used for forming the device(s). FIG. 2A shows a polypropylene mesh (PPKM601) having a pore size of 0.5 mm and monofilament thickness of 0.3 mm; FIG. 2B shows a polypropylene mesh (PPKM602) having a pore size of 0.53 mm and monofilament thickness of 0.18 mm; FIG. 2C shows a polypropylene mesh (PPKM404) having a pore size of 0.53 mm and monofilament thickness of 0.13 mm; and FIG. 2D shows a polypropylene mesh (PPKM604) having a pore size of 0.85 mm and monofilament thickness of 0.2 mm.

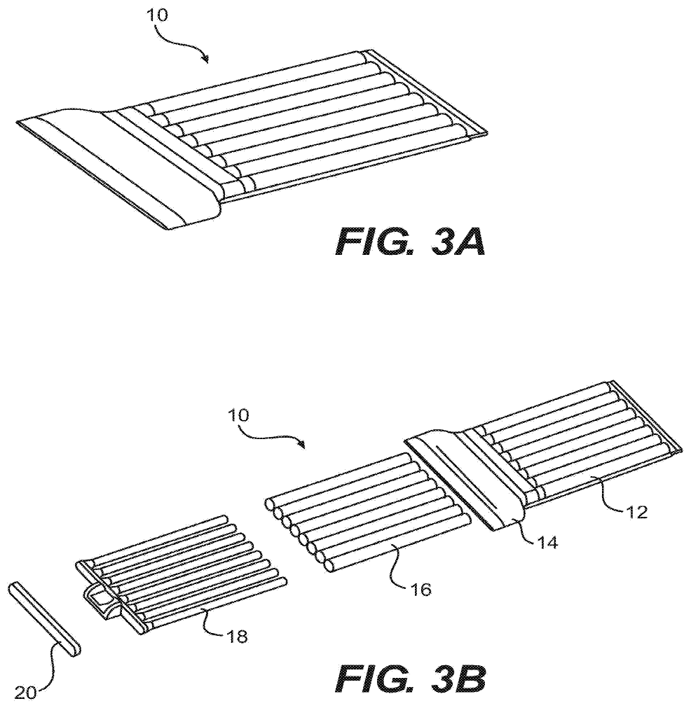

FIG. 3A shows an embodiment of an exemplary device 10. FIG. 3B shows components of the device 10. Device 10 comprises a porous scaffold 12, a primary seal 14, at least one plug system comprising an outer plug 16 and an inner plug 18, and a secondary seal 20.

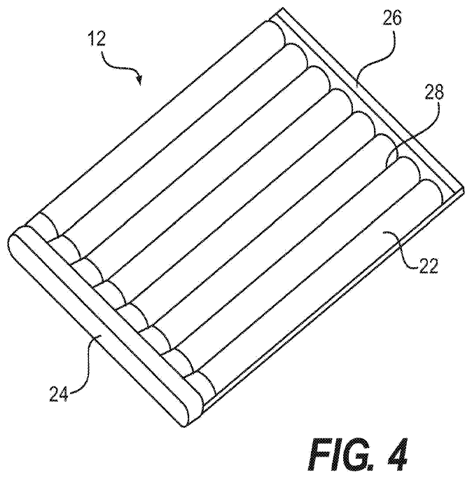

As shown in FIG. 4, porous scaffold 12 of cell transplantation device 10 may comprise a polymer mesh (e.g., a polypropylene mesh, a PTFE mesh, or any other suitable material) that forms one or more porous chambers 22 for containing cells in a host body. In some embodiments, the porous scaffold 12 may comprise 1, 2, 3, 4, 5, 6, 7, 8, 9, 10, 11, 12 or more porous chambers 22. In some embodiments, the availability of multiple chambers allows the use of any number or combination of chambers depending on the volume of cellular preparation required, which is within the knowledge and skill of persons skilled in the art to determine.

Porous chambers 22 may be created, e.g., by joining the top and bottom surfaces of porous scaffold 12 along a line substantially parallel to a longitudinal axis of the device. Multiple porous chambers 22 may have equal or different cross-sectional dimensions and surface areas. In some embodiments, multiple porous chambers 22 are formed by ultrasonically welding the polymer mesh from a proximal end 24 to a distal end 26 of the scaffold. In some embodiments, the top and bottom surfaces of porous scaffold 12 are continuous across the one or more porous chambers 22, interrupted only by ultrasonic weld lines 28 that run substantially parallel to a longitudinal axis of porous scaffold 12. In some embodiments, the top and bottom surfaces of porous scaffold 12 can be indented slightly at each weld line, e.g., to offer additional surface area for vascularization and provides physical stability to device 10 within a host. In some embodiments, the edges at distal end 26 are tapered and ultrasonically welded to one another to seal the distal end 26.

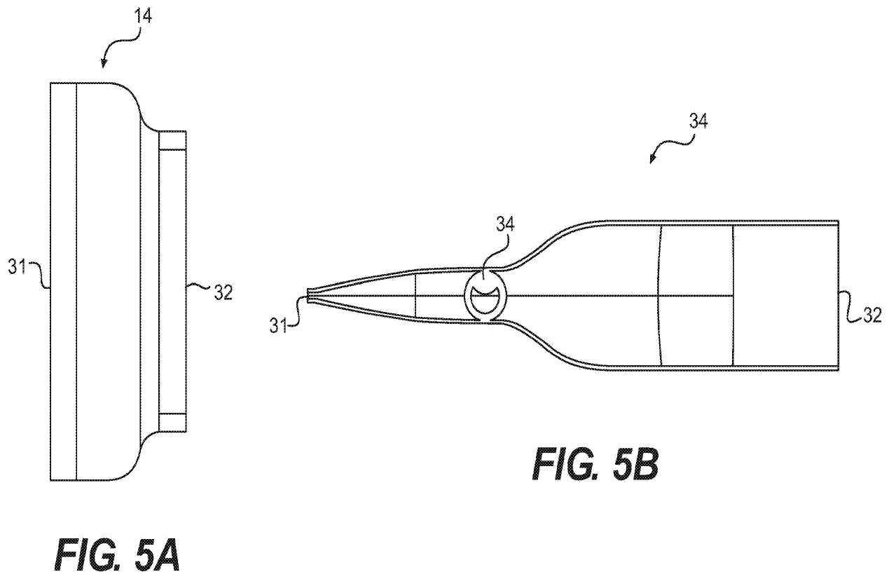

With reference to FIG. 3B, in some embodiments, primary seal 14 is configured to seal the one or more porous chambers 22 during device incubation and after cell infusion. In some embodiments, primary seal 14 comprises an inert and biocompatible polymeric film or any other suitable material. In some embodiments, primary seal 14 is ultrasonically welded at the lateral edges and at the tapered proximal end 31, as shown in FIGS. 5A-5B. In some embodiments, distal end 32 of primary seal 14 is attached to proximal end 24 of porous scaffold 12. In some embodiments, distal end 32 is ultrasonically welded to proximal end 24 of porous scaffold 12.

In some embodiments, primary seal 14 comprises a re-sealable lock 34, e.g., to assist in maintaining the at least one outer plug 16 within a porous chamber 22 during the incubation period. Lock 34, in some embodiments, also prevents leakage of cellular preparation during the cell infusion process. Any suitable re-sealable locking mechanism may be used as lock 34. In some embodiments, lock 34 comprises interlocking groove and ridge features that form a tight seal when pressed together, and unlocks when the top and bottom surfaces of seal 14 are pulled apart at the proximal end 31. Following the device incubation period, in some embodiments, access to outer plug 16 is achieved by trimming proximal end 31 of primary seal 14 and opening re-sealable lock 34. After the cell preparation is delivered into porous scaffold 12, in some embodiments, lock 34 is reclosed and proximal end 31 is re-sealed using, e.g., surgical sutures, staples or bio-adhesives, or hermetic seals.

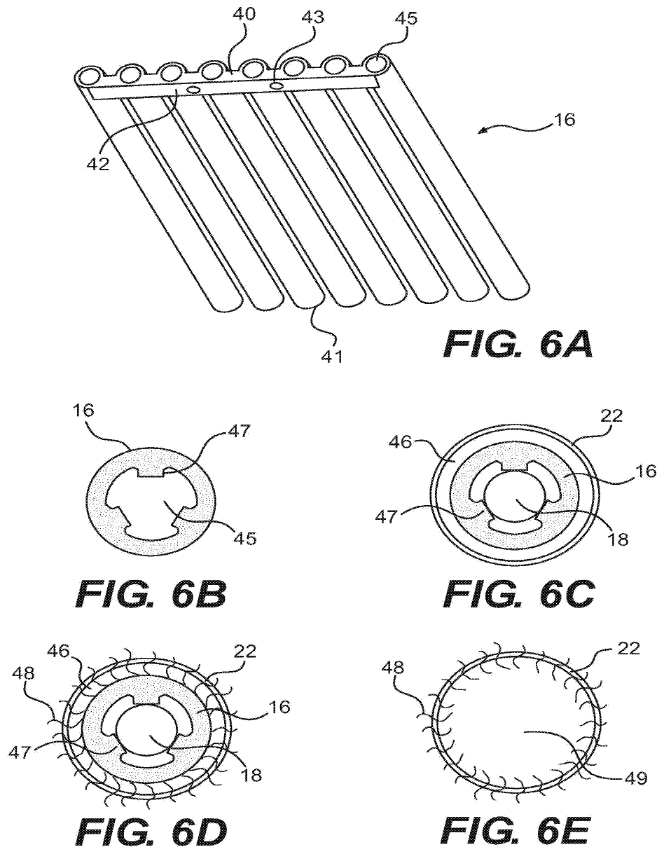

The number of plug systems may correspond to the number of porous chambers 22 in cell transplantation device 10. In some embodiments, outer plug 16 is housed within porous chamber 22 during the device incubation period. In some embodiments, the length of outer plug 16 is approximately equal to the length of the respective porous chamber 22. As illustrated in FIG. 6A, in some embodiments, multiple outer plugs 16 are connected at a proximal end 40 using a common spine 42. Common spine 42 may include one or more grooves 43 to facilitate removal of outer plugs 16 from porous chambers 22. For example, grooves 43 may allow common spine 42 to be grasped using forceps.

In some embodiments, outer plug 16 has a hollow core 45 that houses an inner plug 18. As shown in FIG. 6B, in some embodiments, hollow core 45 is constrained with one or more internal bosses 47 along the length of the inner surface of the plug. In some embodiments, internal bosses 47 provide an air space between the outer plug 16 and the inner plug 18, e.g., to allow trapped air bubbles to escape during the delivery of the cellular preparation. The air space may also prevent vacuum formation during the removal of inner plug 18, and thereby maintain the integrity of the newly formed vascularized collagen matrix in and around the porous chamber. Thus, in some embodiments, the plug system comprising outer plug 16 and inner plug 18 may facilitate delivery of cells to the cell transplantation device 10, and/or increase the chances of cell survival within an intact collagen matrix.

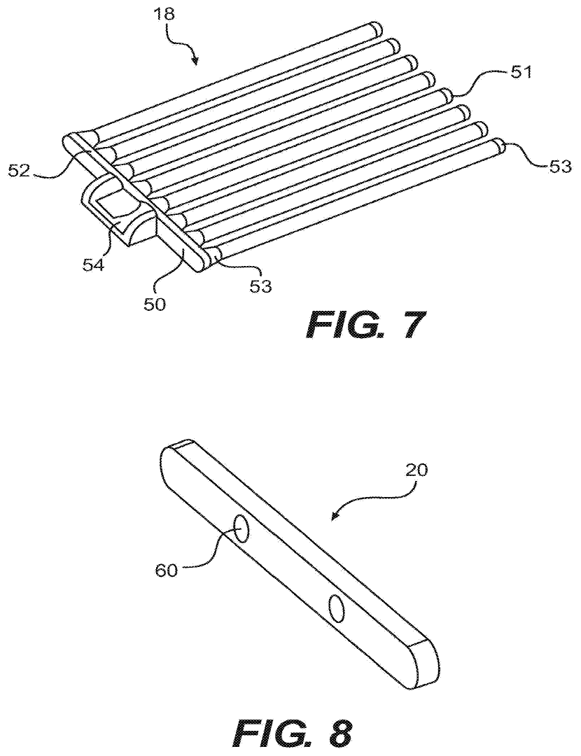

In some embodiments, proximal end 40 and distal end 41 of outer plug 16 comprise sealing mechanisms, such as internal grooves or tapered surfaces, e.g., to ensure an effective seal with inner plug 18. As shown in FIG. 7, proximal end 50 and distal end 51 of inner plug 18 may include complementary sealing mechanisms 53 to prevent infiltration of collagen matrix into hollow core 45 during the incubation period. For example, in some embodiments, sealing mechanism 53 comprises a groove extending around the periphery of the proximal and distal ends of inner plug 18, and outer plug 16 comprises a ridge around the periphery of its distal and proximal ends. In some embodiments, the ridge on outer plug 16 and the groove on inner plug 18 may interlock when inner plug 18 is inserted into the hollow core 45 of outer plug 16, so as to form a complete seal between the inner and outer plugs and/or prevent permeation of any biological material into hollow core 45. Additionally, in some embodiments, if outer plug 16 comprises one or more internal bosses 47, the height of the ridges at the proximal and distal ends of outer plug 16 may be greater than the height of the internal bosses 47.

FIGS. 6C-6D show cross-sectional views of porous chamber 22 and plug 16, 18 assembly, in accordance with embodiments of the present disclosure. FIG. 6C is a cross-sectional view of the assembly prior to implantation in a host body; and FIG. 6D is a cross-sectional view of the assembly after incubation in a host body. In some embodiments, the inner diameter of porous chamber 22 and outer diameter of outer plug 16 are selected to maintain a space 46 around the periphery of outer plug 16 for tissue formation. For example, in some embodiments, the inner diameter of porous chamber 22 is no greater than 4.5 mm and the outer diameter of plug 16 is no greater than 3.5 mm. In some embodiments, the inner diameter of porous chamber 22 is no greater than 3.5 mm and the outer diameter of plug 16 is no greater than 2.5 mm. These embodiments may provide, for example, approximately 0.5 mm of space around outer plug 16 for formation of a vascularized collagen matrix. The space around outer plug 16 may also offer sufficient room for insertion and retraction of the outer plug into and out of the porous chamber.

When cell transplantation device 10 is implanted in a host body, in some embodiments, vascular and connective tissues can penetrate through porous chamber 22 into space 46 and form a vascularized tissue matrix 48 around outer plug 16. In some embodiments, plug 16 prevents penetration of tissue matrix 48 further into the lumen of porous chamber 22. When inner plug 18 and outer plug 16 are retracted from porous chamber 22, in some embodiments, a pocket 49 is created within porous chamber 22, which may be used for containing cells in the host body. Pocket 49 can be enveloped in vascularized tissue matrix 48, as shown in FIG. 6E.

The number of inner plugs 18 may correspond to the number of outer plugs 16. Inner plug 18 may be housed within hollow core 45 of outer plug 16 during the device incubation phase. In some embodiments, multiple inner plugs 18 are connected at a proximal end 50 using a common spine 52. In some embodiments, common spine 52 comprises a clip feature 54 to assist in the handling of inner plug 18 during extraction from outer plug 16.

Secondary seal 20, as shown in FIG. 8, may be used to contain the cellular preparation in the porous chambers when the primary seal 14 is reclosed after delivery of a cell preparation into the cell transplantation device 10. In some embodiments, secondary seal 20 is positioned at proximal end 24 of porous scaffold 12 after the cell preparation is completely delivered into porous chamber 22 and outer plug 16 is retracted from device 10. In some embodiments, secondary seal 20 comprises grooves 60 to facilitate insertion into device 10 using tweezers.

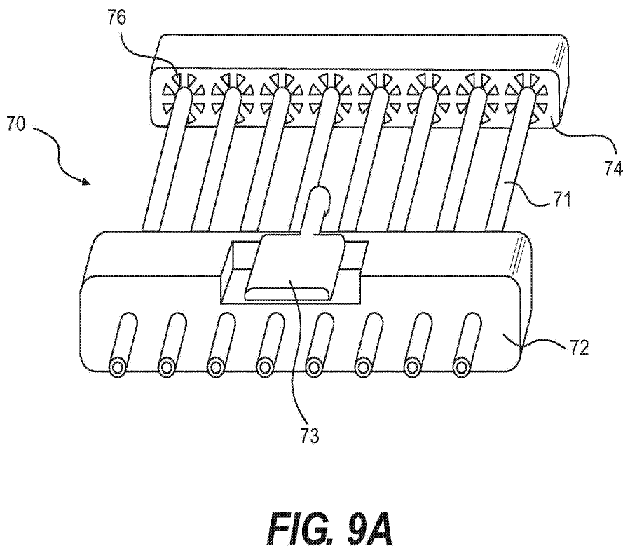

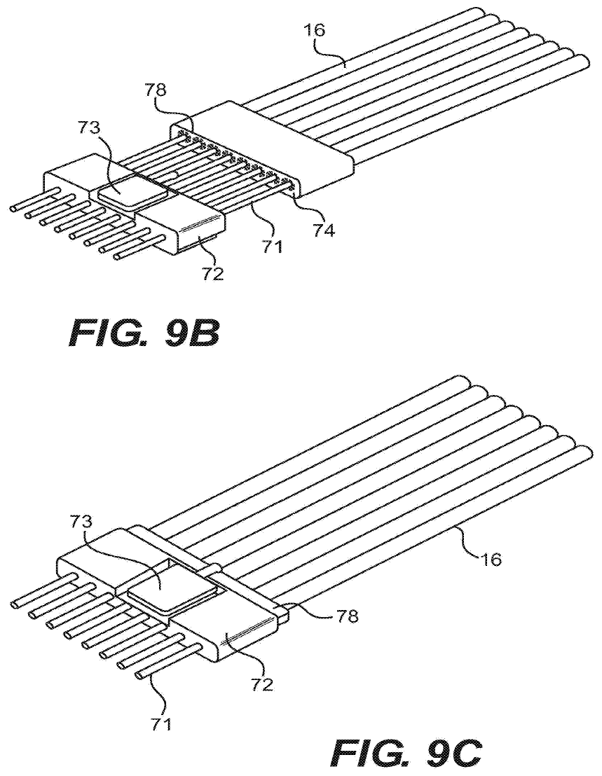

In some embodiments, the device used in the methods described herein further comprises a cell delivery device, and will be explained with reference to cell transplantation device 10. FIG. 9A shows various components of a cell delivery device 70. The cell delivery device 70 comprises at least one cell infusion tube 71, connector cap 72 having a clip feature 73, and connector spacer 74.

Cell infusion tube 71 may comprise polymeric tubing (e.g. polyethylene tubing) or any other suitable material to deliver the cell preparation into porous chamber 22 of device 10 during the cell infusion step. The number of cell infusion tubes in the delivery system may correspond to the number of porous chambers 22.

In some embodiments, connector spacer 74 is positioned at the distal end of cell infusion tube 71 and couples and/or interfaces with the proximal end 40 of outer plug 16 during the cell delivery process. Connector spacer 74 may include one or more through-holes through which cell infusion tube 71 is inserted, as shown in FIG. 9A. In some embodiments, the through-holes are configured to provide a light interference fit with cell infusion tube 71. In some embodiments, the fitting is adapted to keep cell infusion tube 71 in place during the cell infusion process. Additionally, in some embodiments, connector spacer 74 comprises vents 76 to expel air from the air spaces in outer plug 16 created by internal boss 47 during the cell delivery process. In some embodiments, outer plug 16 comprises a hub 78 at the proximal end 40. In some embodiments, connector spacer 74 may be inserted into hub 78 during the cell infusion process, e.g., to secure the delivery device 70 to the cell transplantation device 10.

In some embodiments, the proximal end of cell infusion tube 71 comprises connector cap 72. As the tube is inserted into outer plug 16, in some embodiments, connector cap 72 advances distally towards connector spacer 74. When tube 71 is completely inserted into outer plug 16, in some embodiments, connector cap 72 fits over connector spacer 74 and/or hub 78, and clip feature 73 connects with outer plug 16/or hub 78 along common spine 42, as shown in FIG. 9C. In some embodiments, this enables connector cap 72, connector spacer 74, and outer plug 16 to be retracted as a single unit as the cell preparation is infused into porous chamber 22.

In some embodiments of the methods described herein, cellular transplantation is performed, and will be explained with reference to device 10 and cell delivery device 70. The cell transplantation method is not limited to the device embodiments disclosed herein and may be used with any cell transplantation and cell delivery devices.

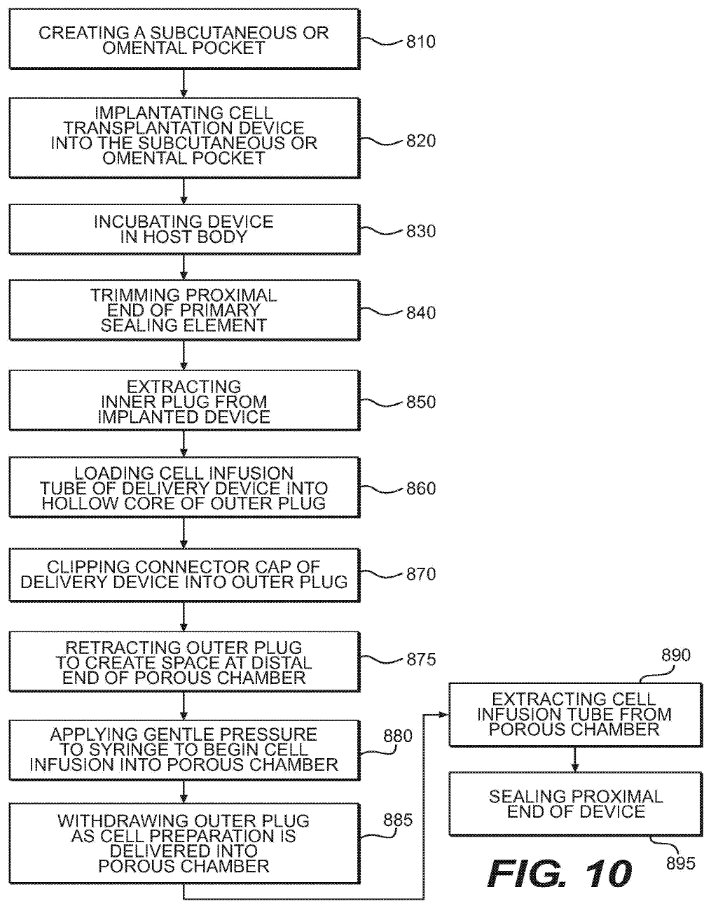

FIG. 10 is a flowchart showing the steps of an exemplary cell transplantation procedure. The cell transplantation procedure is generally a two-step process comprising a device implantation step followed by a cell infusion step. In some embodiments, device 10 is implanted in the host body prior to delivery of cells, e.g., to allow adequate time for collagen and blood vessels to infiltrate porous scaffold 12. In some embodiments, device 10 is sterilized using ethylene oxide prior to implantation. In some embodiments, device 10 may be packaged in a self-seal package or any other sterilizable package along with a sterility indicator strip for an ethylene oxide-based sterilization process. In some embodiments, gamma radiation or dry heat autoclaving is used to sterilize the device prior to implantation. The type of sterilization method used is typically dependent on the scaffold material, since dry heat autoclaving is known to warp certain polymeric materials (e.g., polypropylene) due to low heat deflection temperature. In some embodiments, gamma radiation, at a sterilization dose of 6 M-Rad, can sterilize cell implantation devices; however, gamma radiation may decrease the shelf life of devices made of polypropylene.

Device 10 may be implanted subcutaneously or intraperitoneally. For example, for subcutaneous implantation of the device in the host body, an incision may be made through the dermis and epidermis followed by careful blunt dissection of connective tissue and adipose, creating a subcutaneous pocket caudal to the incision line (step 810). In some embodiments, once an adequate space is created (roughly the dimensions of the device), device 10 is implanted into the subcutaneous pocket, and the incision is sutured (step 820). Alternatively, in some embodiments, device 10 may be implanted in the peritoneal cavity through an abdominal incision. In some embodiments, the device implantation steps (steps 810 and 820) are followed by a device incubation period (step 830) during which a vascularized collagen matrix is deposited in and around porous scaffold 12.

After the incubation period, in some embodiments, device 10 is accessed through a second surgical incision. For example, proximal end 31 of primary seal 12 may be trimmed in situ to open device 10 (step 840). In some embodiments, inner plug 18 is then extracted from outer plug 16 and discarded (step 850). During the inner plug removal process, in some embodiments, air movement is facilitated by internal bosses 47, e.g., to prevent formation of a vacuum inside the device, which can cause disruption of any newly formed blood vessels in and around the device. In some embodiments, removal of inner plug 18 disengages proximal end 50 and distal end 51 of inner plug 18 from proximal end 40 and distal end 41 of outer plug 16. In some embodiments, a cellular preparation is then delivered into device 10 using cell delivery device 70.

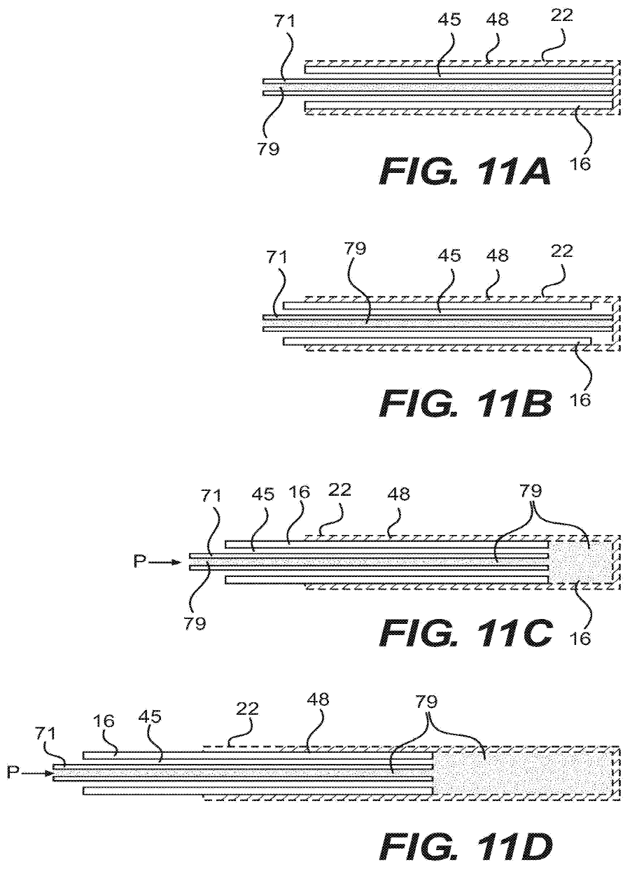

FIGS. 11A-11D show a schematic overview of certain steps of an exemplary cell infusion procedure, and will be explained with reference to the flowchart shown in FIG. 10. For administering the cells into device 10, in some embodiments, cell infusion tube 71 of delivery device 70 is loaded with cellular preparation 79, and the tube is inserted into the hollow core 45 of outer plug 16, as shown in FIG. 11A (step 860). In some embodiments, connector spacer 74 couples with the proximal end 41 and/or hub 78 of outer plug 16. As tube 71 is advanced into the outer plug, in some embodiments, air is vented through internal bosses 47 of outer plug 16 and vents 76 of connector spacer 74. When tube 71 is advanced all the way into outer plug 16, in some embodiments, connector cap 72 interfaces with connector spacer 74. In some embodiments, clip 73 of connector cap 72 is then connected to hub 78 of outer plug 16 (step 870). In some embodiments, outer plug 16, connector cap 72 and connector spacer 74 are then retracted slightly from porous chamber 22 as a single unit, e.g., to create a space at the distal end of porous chamber 22 (step 875). In some embodiments, outer plug 16 may be retracted slightly from porous chamber 22 prior to connecting delivery device 70 with outer plug 16. That is, in some embodiments, step 875 may be performed prior to step 870. In some embodiments, gentle pressure is applied to a syringe connected to cell infusion tube 71 to deliver the cells into porous chamber 22 (step 880). Care is taken to ensure tube 71 remains in the porous chamber 22 as pressure is applied to deliver the cellular preparation.

In some embodiments, outer plug 16 is retracted approximately 5 mm before the cell infusion is started, as shown in FIG. 11B. As pressure (P) is applied to the syringe connected to cell infusion tube 71, in some embodiments, the cell preparation 79 infuses into the porous chamber 22. As the cell preparation is delivered into porous chamber 22, in some embodiments, outer plug 16 and cell infusion tube 71 are withdrawn from the device, as shown in FIGS. 11C-11D (step 885). In some embodiments, when the device is completely filled with the cellular preparation 79, cell infusion is stopped and cell infusion tube 71 is completely retracted from device 10 (step 890). In some embodiments, porous chamber 22 is then evaluated for remaining capacity for cellular preparation, and any remaining cell preparation may be carefully added to the end of the porous chamber. In some embodiments, the cell preparation is contained within the porous chamber 22 by placing secondary seal 20 at the proximal end 40 of porous chamber 22, followed by closing the re-sealable lock 34 of primary seal 12, and securing the proximal end 31 of primary seal 12 with surgical sutures or staples or other suitable sealing mechanisms (step 895). Finally, in some embodiments, the surgical incision is closed using surgical sutures, staples or tissue adhesives, thereby completing the cell transplantation procedure.

In some embodiments, the devices and methods of cell transplantation discussed herein are used for transplantation of cells, or a combination of cells, into a host body to provide therapeutic biological material to the host, e.g., for the treatment of a disease, disorder, or condition (e.g., diabetes). In some embodiments, the devices and methods of cell transplantation discussed herein are used for transplantation of cells, or a combination of cells, into a host for treating diabetes in a host, e.g., a patient in need thereof. Therapeutic methods using the described devices and methods of cell transplantation are provided herein.

An exemplary embodiment is a method of treating diabetes in a patient in need thereof, comprising:
implanting a device in the patient, wherein the device comprises:
   a porous scaffold comprising an immunologically compatible polymer mesh forming the walls of at least one chamber, wherein the chamber comprises an opening at either or both of a proximal end and a distal end of the chamber, wherein the proximal end and the distal end are separated by a lumen that is bounded by the walls, and wherein the porous scaffold has pores sized to facilitate growth of vascular and connective tissues around and through the walls of the at least one chamber;
   at least one removable, non-porous plug configured to be positioned within the lumen of the at least one chamber, wherein the plug extends along the lumen of the chamber; and
   at least one seal configured to enclose either or both the proximal end and the distal end of the chamber;
maintaining the device in the patient's body until the device is infiltrated with vascular and connective tissues;
accessing the implanted device;
withdrawing the plug; and
infusing the chamber with cells, wherein at least some of the cells express insulin, and wherein the insulin-expressing cells are administered at a borderline mass of about 3000 IEQ/kg or greater, preferably suspended in blood, e.g., plasma and/or serum, from the patient.

As used herein, the term “treat,” “treating,” or “treatment” refers to any improvement of any consequence of a disease, disorder, or condition, such as prolonged survival, less morbidity, and/or a lessening of side effects which result from an alternative therapeutic modality. In some embodiments, treatment comprises delaying or ameliorating a disease, disorder, or condition (i.e., slowing or arresting or reducing the development of a disease or at least one of the clinical symptoms thereof). In some embodiments, treatment comprises delaying, alleviating, or ameliorating at least one physical parameter of a disease, disorder, or condition, including those which may not be discernible by the patient. In some embodiments, treatment comprises modulating a disease, disorder, or condition, either physically (e.g., stabilization of a discernible symptom), physiologically (e.g., stabilization of a physical parameter), or both. In some embodiments, treatment comprises transplantation of cells (e.g., therapeutic cells, e.g., islets) to a patient (e.g., a diabetic patient), to obtain a treatment benefit enumerated herein. The treatment can be to cure, heal, alleviate, delay, prevent, relieve, alter, remedy, ameliorate, palliate, improve, or affect a disease, disorder, or condition (e.g., diabetes), the symptoms of a disease, disorder, or condition (e.g., diabetes), or a predisposition toward a disease, disorder, or condition (e.g., diabetes). In some embodiments, the disease, disorder, or condition needing treatment is diabetes (e.g., type 1 diabetes). In some embodiments, the disease, disorder, or condition needing treatment is type 1 diabetes (T1DM).

The terms “subject” and “patient” are used interchangeably herein to refer to any human or non-human animal. Non-human animals include all vertebrates (e.g., mammals and non-mammals) such as any mammal. Non-limiting examples of mammals include humans, mice, rats, rabbits, dogs, monkeys, and pigs. In some embodiments, the patient is a human. In some embodiments, the patient is a human with diabetes (e.g., type 1 diabetes).

The term “a patient in need of treatment,” as used herein, refers to a patient that would benefit biologically, medically, or in quality of life from a treatment (e.g., a treatment using any of the exemplary methods described herein).

As used herein, the term “diabetes” or “diabetes mellitus” refers to a medical condition characterized by elevated levels of plasma glucose (hyperglycemia) in the fasting state. Chronic diabetes conditions include type 1 diabetes and type 2 diabetes. In type 1 diabetes (T1DM), or insulin-dependent diabetes mellitus (IDDM), patients can produce little or no insulin (i.e., the hormone capable of regulating glucose utilization). In type 2 diabetes (T2DM), or noninsulin-dependent diabetes mellitus (NIDDM), patients can produce insulin and may even exhibit hyperinsulinemia (i.e., plasma insulin levels that are the same or elevated in comparison with non-diabetic subjects), while at the same time demonstrating hyperglycemia. Potentially reversible diabetes conditions include prediabetes (i.e., a condition in which plasma glucose levels may be higher than normal, but not high enough to be classified as diabetes), as well as gestational diabetes (i.e., a condition which can occur during pregnancy, but resolves after the baby is delivered). In some embodiments, the diabetes is type 1 diabetes. In some embodiments, the diabetes is type 1 diabetes mellitus with hypoglycemia unawareness.

In some embodiments of the methods disclosed herein, the patient has type 1 diabetes mellitus with hypoglycemia unawareness. In some embodiments, the patient has type 1 diabetes mellitus with hypoglycemia unawareness as measured by i) a Clarke reduced awareness score of 3, 4, 5, or more (e.g. about 4 or more); ii) a HYPO score greater than or equal to the 90th percentile (e.g., about 1047) during the screening period and within the last 6 months; iii) one or more swings in blood glucose despite diabetes therapy as defined by an LI score greater than or equal to the 90th percentile (e.g., about 433 mmol/L²/h wk⁻¹) during the screening period and/or within the 6 months prior to treatment; and/or iv) a composite Clarke score of about 4 or more and a HYPO score greater than or equal to about the 75th percentile and a LI greater than or equal to about the 75th percentile (e.g., about 329) during the screening period and/or within the 6 months prior to treatment. In some embodiments, the patient has a history of severe hypoglycemic episodes. In some embodiments, the patient has required insulin for about 2, 3, 4, 5, 6, 7, 8, 9 or 10 or more years (e.g., at least about 5 years). In some embodiments, the patient is between about 18 and 65 years of age.

As used herein, the term “severe hypoglycemia” or “severe hypoglycemic episode” encompasses at least one event with at least one of the following symptoms: memory loss; confusion; uncontrollable behavior; irrational behavior; unusual difficulty in awakening; suspected seizure; seizure; loss of consciousness; or visual symptoms, in which the patient was unable to treat him/herself and which was associated with either a blood glucose level <54 mg/dL [3.0 mmol/L] or prompt recovery after oral carbohydrate, IV glucose, or glucagon administration. In addition, in some embodiments, composite indices of hypoglycemia frequency, severity, and/or symptom recognition may be assessed, e.g., to determine a severe hypoglycemic episode, by a Clarke survey and/or HYPO score.

In some embodiments, a Clarke survey (as described in, e.g., Clarke et al. (1995) Care 18 (4): 517-22, which is incorporated herein by reference) is used alone or in combination with another method (e.g., HYPO score) to assess awareness of hypoglycemia. In some embodiments, a Clarke survey involves subject completion of eight questions characterizing the subject's exposure to episodes of moderate and severe hypoglycemia. In some embodiments, a Clarke survey further evaluates the subject's glycemic threshold for, and symptomatic responses to, hypoglycemia. I In some embodiments, the eight question survey is scored according to an answer key that gives a total score between 0 and 7 (most severe). In some embodiments, a score of 4 or more indicates reduced and/or impaired awareness of hypoglycemia and/or increased risk for severe hypoglycemic events.

In some embodiments, a HYPO score (as described, e.g., in Ryan et al. (2004) Diabetes 53 (4): 955-62, which is incorporated herein by reference) is used alone or in combination with another method (e.g., Clarke survey) to assess awareness of hypoglycemia. In some embodiments, a HYPO score involves subject recording of blood glucose readings and hypoglycemic events (blood glucose (BG) <3.0 mmol/L [54 mg/dL]) over a 4-week period and recall of all severe hypoglycemic episodes in the previous 12months. In some embodiments, calculation of a HYPO score comprises at least 28 days of capillary blood glucose tests within a 35-day period with at least four tests per day. In some embodiments, a HYPO score greater than or equal to the 90th percentile (1047) of values derived from an unselected group of type 1 diabetes patients indicates severe problems with hypoglycemia.

In some embodiments, a mixed meal tolerance test (MMTT) is performed on a patient, e.g., prior to treatment, e.g., to assess islet function and/or determine basal (fasting), stimulated glucose, and/or C-peptide levels. In some embodiments, the patient has a blood glucose level of 70-180 mg/dl (3.89-10 mmol/L) prior to the meal test. In some embodiments, basal glucose and/or C-peptide levels in the patient are determined prior to the meal test. In some embodiments, the meal test comprises administration of a standardized meal to the patient. An exemplary standardized meal is Boost® High Protein Drink (or a nutritionally equivalent substitute). In some embodiments, the standardized meal is and/or the meal test comprises Boost® 6 mL/kg body weight (to a maximum of 360 mL). In some embodiments, the patient is administered Boost® 6 mL/kg body weight (to a maximum of 360 mL) to consume in, e.g., about 5 minutes, starting at time 0. In some embodiments, blood samples for stimulated glucose, C-peptide, and/or insulin levels are then drawn from the patient (e.g., at time 15, 30, 45, 60, 90, 120, 180, and/or 240 minutes). In some embodiments, the standardized meal and/or meal test provides up to 50 carbohydrates without insulin treatment during an about 1, 2, 3, 4, or 5 hour test (e.g., an about 2 or 4 hour test). In some embodiments, the standardized meal and/or meal test causes short term hypoglycemia in the patient. In some embodiments, immediately after the meal test is completed, an adequate insulin dose is administered to the patient, e.g., to ensure proper glycemic control.

In some embodiments of the methods disclosed herein, the patient has less than about 0.3 ng/ml C-peptide in a serum sample prior to treatment in response to a mixed meal tolerance test (e.g., a meal test using Boost® 6 mL/kg body weight to a maximum of 360 mL). In some embodiments, the patient has less than about 0.3 ng/ml C-peptide in a serum sample prior to treatment in response to a mixed meal tolerance test (e.g., a meal test using Boost® 6 mL/kg body weight to a maximum of 360 mL) as measured during an about 1, 2, 3, 4, or 5 hour test (e.g., an about 2 hour test). In some embodiments, the patient is restored to normoglycemia.

In some embodiments, a patient is assessed for glycemic control and/or glycemic lability. The term “glycemic control,” as used herein, refers to the typical levels of blood glucose in a subject with diabetes, e.g., type 1 diabetes. Exemplary glycemic biomarkers that may be used to monitor glycemic control include but are not limited to: glycated hemoglobin (i.e., hemoglobulin A1c (HbA1c)), fructosamine, glycated albumin (GA), and 1,5-anhydroglucitol (1,5-AG). In some embodiments, glycemic control is monitored in a patient, e.g., by measuring levels of HbA1c and/or one or more alternate glycemic biomarkers.

The term “glycemic lability,” as used herein, refers to a lability index (LI) calculated based on change in a patient's glucose levels over time. Typically, a LI requires 4 or more daily capillary blood glucose measurements over a 4-week period, and is calculated as the sum of all the squared differences in consecutive glucose readings divided by how far apart the readings were determined (range 1 to 12 hours) in mmol/L²/h wk⁻¹. In some embodiments, a LI greater than or equal to the 90th percentile (433 mmol/L²/h wk⁻¹) of values derived from an unselected group of type 1 diabetes patients indicates severe glycemic lability.

In some embodiments of the methods disclosed herein, at least some of the cells infused in the chamber express insulin. In some embodiments, the cells and/or the insulin-expressing cells are administered at a borderline mass of about 3000, 3100, 3200, 3300, 3400, 3500, 3600, 3700, 3800, 3900, or 4000 IEQ/kg, or greater. In some embodiments, the cells and/or the insulin-expressing cells are administered at a borderline mass of about 4000, 4100, 4200, 4300, 4400, 4500, 4600, 4700, 4800, 4900,or 5000 IEQ/kg, or greater. In some embodiments, at least about 60%, 65%, 70%, 75%, 80%, 85%, or 90% (e.g., at least 70%) of the cells infused in the chamber are purified islets. In some embodiments, at least about 70%, 75%, 80%, 85%, or 90% (e.g., at least 80%) of the cells infused in the chamber are viable islets.

As used herein, the term “IEQ” or “islet equivalent,” refers to volume of islet cells equal to that of a sphere having a 150 μm diameter. The term “IEQ/kg” refers to the number of islet equivalents administered per kilogram of a patient's body weight.

As used herein, the term “purified islet” refers to one or more than one islet cell substantially free from other tissue naturally found around the islets, e.g., acinar tissue. In some embodiments, at least about 70% of the cells infused in the chamber are purified islets. Without wishing to be bound by theory, a large amount of acinar tissue surrounding islets may affect islet engraftment when competing for oxygen and nutrients, may result in the release of pancreatic enzymes, and/or may potentiate islet damage and/or inflammatory reactions. In some embodiments, islets with >70% purity are infused into the chamber. In some embodiments, islets with >90% purity are infused into the chamber. In some embodiments, islets with 70-90% purity are infused into the chamber. In some embodiments, islets with different purities are infused into separate chambers of the device.

Methods of quantifying the number or percentage of islets in an islet preparation are known in the art and described in, e.g., Papas et al. (2009) Curr Opin Organ Transplant 14 (6): 674-82. An exemplary method is manual, visual counting of islet equivalents (IEQ) under a light microscope following dithizone (DTZ) staining to determine the total volume of islet tissue and its purity. Exemplary methods for estimating the total number of cells and/or volume of tissue in a preparation include measurements of intracellular deoxyribonucleic acid (DNA), cellular nuclei counts, large particle flow cytometry, and packed tissue volume; however, such methods may not provide islet- or β-cell specific information, so may require an independent estimate of the purity (fractional volume of islet tissue or β-cells). Such estimates can be obtained using a variety of methods, including but not limited to: morphological analysis with electron and/or light microscopy, immunohistochemistry with laser scanning confocal microscopy, and laser scanning cytometry (see, e.g., Colton et al. (2007) Cellular Transplantation: From Laboratory to Clinic. 85-134).

As used herein, the term “viable islet” refers to one or more than one islet cell with sufficient membrane integrity as determined by, e.g., a dye exclusion assay. An exemplary dye exclusion assay is fluorescein diacetate/propidium iodide (FDA/PI) (see, e.g., Papas et al. (2009) Curr Opin Organ Transplant 14 (6): 674-82). In some embodiments, at least about 80% of the cells infused in the chamber are viable islets.

In some embodiments, the cells are purified from a pellet prior to administration. In some embodiments, the pellet has a volume of less than about 30, 25, 20, 15, 12.5, 10.5, or 7.5 ml (e.g., less than about 10.5 ml). In some embodiments, the pellet has a volume of less than about 10.5 ml. In some embodiments, the cells are infused in the chamber about 4-24 weeks after implanting the device. In some embodiments, the cells are infused in the chamber about 4, 5, 6, 7, 8, 9, or 10 weeks after implanting the device. In some embodiments, the cells are infused in the chamber about 6 weeks after implanting the device.

In some embodiments, the methods disclosed herein further comprise administering immunosuppression prior to and/or after infusing the chamber with cells. In some embodiments, the immunosuppression is administered prior to infusion. In some embodiments, the immunosuppression is administered for at least 3, 4, 5, 6, 7, 8, 9, or 10 weeks prior to infusion. In some embodiments, the immunosuppression is administered for at least 3, 4, 5, or 6 weeks prior to infusion. In some embodiments, the immunosuppression is administered for at least 6 weeks prior to infusion. In some embodiments, the immunosuppression is administered after infusion. In some embodiments, the immunosuppression is administered for about 1, 2, 3, 4, 5, 6, 7, 8, 9, or 10 days after infusion. In some embodiments, the immunosuppression is administered for about 7 days after infusion. In some embodiments, the methods disclosed herein further comprise administering etanercept and/or basiliximab.

Without wishing to be bound by theory, in some embodiments, the immunosuppression may allow the patient to achieve a stable immunosuppressive state prior to cell transplant. In some embodiments, immunosuppression may allow the patient to maintain a stable immunosuppressive state during cell exposure and/or engraftment, thereby limiting the risk of allo- and auto-immunization and rejection. In some embodiments, immunosuppression may allow the patient to avoid cytokine release syndrome, which may develop during and/or after thymoglobulin administration and/or may affect cell engraftment. In some embodiments, immunosuppression may allow the patient to avoid one or more detrimental effects of steroids and/or hyperglycemia on freshly transplanted cells. In some embodiments, steroids may be required for prevention and/or treatment of cytokine release syndrome around thymoglobulin infusion. In some embodiments, the immunosuppression comprises (i) induction therapy prior to cell infusion with thymoglobulin and/or (ii) maintenance therapy comprising one or more of tacrolimus, mycophenolate mofetil, and mycophenolic acid administered after device implantation and/or islet transplantation.

In some embodiments, the immunosuppression comprises induction therapy prior to cell infusion with thymoglobulin. In some embodiments, the administered amount of thymoglobulin is a total dose of about 1-10 mg/kg (e.g., about 6 mg/kg). In some embodiments, the administered amount of thymoglobulin is a total dose of about 6mg/kg. In some embodiments, the total dose of thymoglobulin (e.g., about 6 mg/kg) is administered over 1-10 daily infusions (e.g., at least 4 daily infusions). In some embodiments, the total dose of thymoglobulin (e.g., about 6 mg/kg) is administered over at least 4 daily infusions. In some embodiments, the administered amount of thymoglobulin is a total dose of about 6 mg/kg administered over at least 4 daily infusions.

In some embodiments, the immunosuppression comprises maintenance therapy comprising one or more of tacrolimus, mycophenolate mofetil, and mycophenolic acid administered after device implantation and/or islet transplantation.

In some embodiments, the administered amount of tacrolimus is a dose adjusted upwards daily to a blood level of about 1-10 ng/ml (e.g., about 4-6 ng/ml). In some embodiments, the administered amount of tacrolimus is increased to a blood level of about 7-15 mg/ml (e.g., about 8-10 mg/ml) on the day of cell infusion. In some embodiments, the administered amount of mycophenolate mofetil is about 100-750 mg (e.g., 500 mg). In some embodiments, the administered amount of mycophenolate mofetil is increased to about 500-1500 mg (e.g., about 1000 mg) on the day of cell infusion. In some embodiments, the administered amount of mycophenolic acid is about 100-500 mg (e.g., about 360 mg). In some embodiments, the administered amount of mycophenolic acid is increased to about 500-1000 mg (e.g., about 720 mg) on the day of cell infusion. In some embodiments, administration of tacrolimus, mycophenolate mofetil, and/or mycophenolic acid commences about 1-5 weeks (e.g., about 3-4 weeks) after device implantation. In some embodiments, administration of tacrolimus, mycophenolate mofetil, and/or mycophenolic acid commences about 3-4 weeks after device implantation.

In some embodiments, the methods disclosed herein further comprise screening a patient for islet function after cell infusion by checking for a C-peptide level in a serum sample. In some embodiments, the patient is screened about 1, 2, 3, 4, 5, 6, 7, 8, 9, 10, 11, or 12 months (e.g., about 6 months) after cell infusion by checking for a C-peptide level in a serum sample. In some embodiments, the C-peptide level (e.g., in a serum sample) may be determined one or more times or continuously throughout the period of graft survival in the patient (e.g., about 1 year or more, about 2 years or more, about 5 years or more, about 7 years or more, or about 10 years or more). In some embodiments, the methods disclosed herein further comprise administering a second infusion of cells to a patient having a C-peptide level of less than about 0.2, 0.3, 0.4, or 0.5 ng/ml (e.g., about 0.3 ng/ml). In some embodiments, the methods disclosed herein further comprise administering at least a third or fourth infusion of cells to a patient having a C-peptide level of less than about 0.2, 0.3, 0.4, or 0.5 ng/ml (e.g., about 0.3 ng/ml). In some embodiments, the methods disclosed herein further comprise administering more than four infusions of cells to a patient having a C-peptide level of less than about 0.2, 0.3, 0.4, or 0.5 ng/ml (e.g., about 0.3 ng/ml). In some embodiments, the methods disclosed herein further comprise administering two, three, four, or more infusions of cells to a patient.

In some embodiments, the methods disclosed herein further comprise monitoring a patient for up to one year for insulin-independence after the start of the procedure. In some embodiments, the patient is monitored at about 5, 6, 7, 8, 9, 10, 11, or 12 months after the start of the procedure. In some embodiments, the methods disclosed herein further comprise administering one or more additional infusions of cells to a patient who, at the end of the monitoring period, has a C-peptide level in a serum sample of less than about 0.2, 0.3, 0.4, or 0.5 ng/ml (e.g., about 0.3 ng/ml). In some embodiments, the methods disclosed herein further comprise administering one or more additional infusions of cells to a patient who, at the end of the monitoring period, is not insulin-independent. In some embodiments, the methods disclosed herein further comprise monitoring the patient for up to one year for insulin-independence after the second or subsequent infusion (e.g., after the second, third, or fourth infusion, etc.). In some embodiments, the patient is monitored at about 5, 6, 7, 8, 9, 10, 11, or 12 months after the second or subsequent infusion (e.g., after the second, third, or fourth infusion, etc.). In some embodiments, the methods disclosed herein further comprise monitoring the patient for up to one year for insulin-independence after the third, fourth, or subsequent infusion. In some embodiments, the patient is monitored at about 5, 6, 7, 8, 9, 10, 11, or 12 months after the third, fourth, or subsequent infusion. In some embodiments, the methods disclosed herein further comprise monitoring the patient one or more times or continuously throughout the period of graft survival. In some embodiments, the period of graft survival may be about 1, 2, 3, 4, 5, 6, 7, 8, 9, or 10 years or more (e.g., about 1 year or more, about 2 years or more, about 5 years or more, about 7 years or more, or about 10 years or more).

As used herein, the term “insulin-independent” or “insulin-independence” refers to a patient (e.g., an islet cell recipient) that is able to titrate off insulin therapy for at least 1 week and meets one or more, e.g., all, of the following criteria: (i) fasting capillary glucose level does not exceed 140 mg/dl (7.8 mmol/L) more than three times in 1 week (based on measuring capillary glucose levels a minimum of 7 times in a seven day period); (ii) 2-hours post-prandial capillary glucose does not exceed 180 mg/dl (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/ml (0.16 pmol/L).

As used herein, the term “insulin-dependent” or “insulin-dependence” refers to a patient (e.g., an islet cell recipient) that does not meet the criteria for insulin-independence, as described above.

In some embodiments, the methods disclosed herein comprise implanting 1, 2, 3, 4, 5, 6, 7, 8, 9, 10 devices (e.g., 2-6 devices, e.g., 2-4 devices) in a patient. In some embodiments, the methods disclosed herein comprise implanting 2-6 devices (e.g., 2-4 devices) in the patient. In some embodiments, the device or devices are implanted in the patient's abdomen. In some embodiments, the methods disclosed herein further comprise administering Cephazolin and/or Keflex to the patient. In some embodiments, the methods disclosed herein further comprise a step of imaging the porous scaffold prior to delivering cells.

In some embodiments of the methods disclosed herein, the cells may be allogeneic, xenogeneic or syngeneic donor cells, patient-derived cells, including stem cells, cord blood cells and embryonic stem cells. In some embodiments, stem cells may be differentiated into the appropriate therapeutic cells. In some embodiments, the cells may be immature or partially differentiated or fully differentiated and mature cells when placed into the device. The cells may also be, in some embodiments, genetically engineered cells or cell lines. In some embodiments, transplantation of islets of Langerhans cells is used to provide means for blood glucose regulation in the host body. In some embodiments, co-transplantation of islets of Langerhans and Sertoli cells is used to provide means for blood glucose regulation in the host body. In some embodiments, the Sertoli cells provide immunological protection to the islet cells in the host body. The immune protection provided by Sertoli cells in a host body is described, for example, in U.S. Pat. No. 5,725,854, which is incorporated herein by reference. Accordingly, this disclosure contemplates methods of treating various diseases (e.g., diabetes) by transplanting therapeutic amounts of cells to subjects in need thereof.

In some embodiments of the methods disclosed herein, the cells comprise genetically engineered cells that express insulin. In some embodiments, the cells comprise islets. In some embodiments, the cells comprise islets and/or one or more of Sertoli cells, mesenchymal stem cells, differentiated stem cells, and genetically engineered cells (e.g., genetically engineered cells that express insulin). In some embodiments, the cells comprise stem cells or stem cell-derived cells. In some embodiments, the cells comprise allogeneic, xenogeneic, or syngeneic donor cells, or patient-derived cells. In some embodiments, the cells comprise genetically engineered cells or cell lines. In some embodiments, the cells comprise encapsulated cells. In some embodiments, the cells are encapsulated in alginate, a polysaccharide hydrogel, chitosan, calcium or barium alginate, a layered matrix of alginate and polylysine, photopolymerizable poly (ethylene glycol) polymer, a polyacrylate, hydrogel methacrylate, methyl methacrylate, a silicon capsule, a silicon nanocapsule, a polymembrane, or acrylonitrile-co-vinyl chloride. In some embodiments, the cells comprise two or more cell types selected from islets, Sertoli cells, stem cells, differentiated stem cells, embryonic stem cells, induced pluripotent stem cells, allogeneic cells, xenogeneic cells, syngeneic cells, and genetically engineered cells or cell lines.

In some embodiments, the cells comprise islets and Sertoli cells. In some embodiments, the cells comprise islets and stem cells (e.g., mesenchymal stem cells, differentiated stem cells, induced pluripotent stem cells, and/or embryonic stem cells). In some embodiments, the cells comprise islets and genetically engineered cells. In some embodiments, the cells comprise islets and allogeneic cells. In some embodiments, the cells comprise islets and xenogeneic cells. In some embodiments, the cells comprise islets and syngeneic cells. In some embodiments, the cells comprise islets and encapsulated cells.

In some embodiments, the cells comprise genetically engineered cells that express insulin and Sertoli cells. In some embodiments, the cells comprise genetically engineered cells that express insulin and stem cells (e.g., mesenchymal stem cells, differentiated stem cells, induced pluripotent stem cells, and/or embryonic stem cells). In some embodiments, the cells comprise genetically engineered cells that express insulin and allogeneic cells. In some embodiments, the cells comprise genetically engineered cells that express insulin and xenogeneic cells. In some embodiments, the cells comprise genetically engineered cells that express insulin and syngeneic cells. In some embodiments, the cells comprise genetically engineered cells that express insulin and encapsulated cells. In some embodiments, the cells comprise two or more types of genetically engineered cells (e.g., genetically engineered cells that express insulin and at least one other genetically engineered cell type).

The density of the transplanted therapeutic cells, or combinations of cells, may be determined based on the body weight of the host and/or the therapeutic effects of the cells. In some embodiments, the dimensions of the device and/or number of porous chambers to be used (in a multi-chamber device) is determined based on the number of the cells required, the extent of vascularization achievable during the device incubation period, and/or the diffusion characteristics of nutrients and/or cellular products in and out of the implanted device.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1

### Assessment of Safety, Tolerability, and Efficacy of CP-Device for Clinical Islet Transplantation

To assess the safety, tolerability, and efficacy of islet transplantation into a CP-device (i.e., an exemplary device disclosed herein), the CP-device is implanted under the skin of type 1 diabetes patients (n=7) with hypoglycemia unawareness and a history of severe hypoglycemic episodes. Islets are transplanted into the CP-device a minimum of six weeks after implantation to allow for vascularization of the CP-device chambers and stable immunosuppressive activity of the medication without inflammatory cytokine storm. A subsequent islet transplantation is conducted into previously-implanted, separate CP-devices, if necessary. Islet release criteria that are predictive of clinical transplant outcomes into the CP-device are also established. Clinical transplant outcomes are demonstrated through defined efficacy measures, as described below.

### Procedures

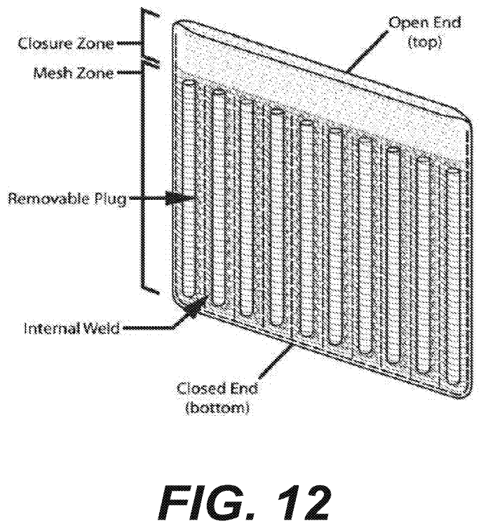

Type 1 diabetes patients are treated by islet transplantation using a subcutaneously implanted CP-device (FIG. 12). Patients with type 1 diabetes (T1DM) characterized by hypoglycemic unawareness and severe hypoglycemic episodes despite optimal insulin treatment, and eligible for pancreatic islet transplantation, are eligible for treatment. Inclusion criteria are set forth in Table 1. Patients who meet any of the exclusion criteria set forth in Table 2 are not eligible for treatment. Patients are treated from enrollment, through islet transplantation and 1 year of follow up (if they achieve insulin independence), or 1 year after intraportal islet transplant (if they do not achieve insulin independence after the first or/and second islet transplant into the CP-device). Treatment may range from approximately 14 months to 33 months from enrollment, depending on the clinical scenario.

A minimum of three weeks after subcutaneous implantation of the CP-device, immunosuppression is initiated and optimized for another 3 weeks. Next, borderline mass (>3,000 IEQ/kg) of purified islets (purity >70%) is implanted in the CP-device. The CP-device is assessed for safety and tolerability. At 90 days post-transplant, a mini CP-device is explanted for further safety and histology assessment. After 180 days, safety, efficacy, and clinical benefit is assessed. This outcome determines if a second islet transplant is performed into additional CP-devices in the patient.

Patients that show no islet function (defined as C-peptide <0.3 ng/ml and no clinical benefit) receive a second CP-device islet transplant and are reset in the safety and efficacy measures. Patients that show partial islet function (defined as C-peptide ≥0.3 ng/ml with no clinical benefit, or C-peptide <0.3 ng/ml and clinical benefit) receive a second CP-device islet transplant and are reset in the safety and efficacy measures. Full function, or insulin-independent, patients are followed up to 1 year post-initial CP-device transplant and reassessed at 1 year. If at one year the patient shows partial function (defined as C-peptide >0.3 ng/ml with no clinical benefit, or C-peptide <0.3 ng/ml and clinical benefit) a second CP-device islet transplant is received and the timing of the safety and efficacy measures are reset. If a patient shows no function (defined as <0.3 ng/ml C-peptide and no clinical benefit) at 180 days post-second transplant, then the CP-devices may be explanted and a subsequent intraportal islet transplant is offered. Since the second islet infusion occurs on average 9 months after the CP-device implantation followed by third islet transplant, but intraportally, after 1 year with another 1 year of follow up, total length of treatment for each patient is expected to be around 33 months.

TABLE 1
Inclusion Criteria

| | |
|---|---|
| 1 | Male and female patients 18 to 65 years of age. |
| 2 | Ability to provide written informed consent. |
| 3 | Mentally stable and able to comply with the procedures of the |
| | treatment protocol. |
| 4 | Clinical history compatible with T1DM with onset of disease at |
| | <40 years of age, insulin-dependence for ≥5 years at the time of |
| | enrollment, and a sum of patient age and insulin dependent |
| | diabetes duration of ≥28. |
| 5 | Absent stimulated C-peptide (<0.3 ng/mL) response to a mixed |
| | meal tolerance test (MMTT; Boost ® 6 mL/kg body weight to a |
| | maximum of 360 mL; another product with equivalent caloric and |
| | nutrient content may be substituted for Boost) measured during a |
| | 2 hour test. |
| 6 | Involvement in intensive diabetes management defined as self- |
| | monitoring of glucose values no less than a mean of three times |
| | each day averaged over each week and by the administration of |
| | three or more insulin injections each day or insulin pump therapy. |
| | Such management must be under the direction of an |
| | endocrinologist, diabetologist, or diabetes specialist with at least 3 |
| | clinical evaluations during the 12 months prior to enrollment. |
| 7 | At least one episode of severe hypoglycemia in the 12 months |
| | prior to enrollment. |
| 8 | Reduced awareness of hypoglycemia as defined by a Clarke score |
| | of 4 or more |
| | OR a HYPO score greater than or equal to the 90th percentile |
| | (1047) during the screening period and within the last |
| | 6 months; |
| | OR marked glycemic lability characterized by wide swings in |
| | blood glucose despite optimal diabetes therapy and defined by an |
| | LI score greater than or equal to the 90th percentile (433 mmol/ |
| | L²/h wk⁻¹) during the screening period and within the last 6 |
| | months prior to randomization; |
| | OR a composite of a Clarke score of 4 or more and a HYPO score |
| | greater than or equal to the 75th percentile (423) and a LI greater |
| | than or equal to the 75th percentile (329) during the screening |
| | period and within the last 6 months. |

TABLE 2
Exclusion Criteria

| | |
|---|---|
| 1 | Body mass index (BMI) >30 kg/m². |
| 2 | Insulin requirement >1.0 IU/kg/day. |
| 3 | Hemoglobin A1c (HbAlc) >11%. |
| 4 | Untreated proliferative diabetic retinopathy. |
| 5 | Blood Pressure: SBP >160 mmHg or DBP >100 mmHg. |
| 6 | Measured glomerular filtration rate <70 mL/min/1.73 m² (using |
| | iohexol or calculated using the subject's measured serum |
| | creatinine and the Chronic Kidney Disease Epidemiology |
| | Collaboration (CKD-EPI equation) or based on 24-hrs urine |
| | collection). Strict vegetarians (vegans) with a calculated GFR |
| | <60 mL/min/1.73 m² are excluded. The absolute (raw) GFR value |
| | is used for subjects with body surface areas >1.73 m². Should this |
| | formula bias CLcr estimate, another more accurate GFR |
| | measurement (e.g., MAG3 or DTPA radioisotope scan, 24 hrs |
| | inuline clearance, etc.) may be used. |
| 7 | Presence or history of macroalbuminuria (>300 mg/g creatinine). |
| 8 | Presence or history of panel-reactive anti-HLA antibodies above |
| | 30%. |
| 9 | For female subjects of child bearing potential: Positive pregnancy |
| | test, presently breast-feeding, or unwillingness to use effective |
| | contraceptive measures for the duration of treatment and 4 months |
| | after discontinuation. For male subjects: intent to procreate during |
| | the duration of treatment or within 4 months after discontinuation |
| | or unwillingness to use effective measures of contraception. Oral |
| | contraceptives, Norplant ®, Depo-Provera ®, and barrier |
| | devices with spermicide are acceptable contraceptive methods; |
| | condoms used alone are not acceptable. |
| 10 | Presence or history of active infection including hepatitis B, |
| | hepatitis C, HIV, or tuberculosis (TB). Subjects with laboratory |
| | evidence of active infection are excluded even in the absence of |
| | clinical evidence of active infection. |
| 11 | Patients who have a negative screen for Epstein Barr Virus by IgG |
| | determination may be included, but will be offered islets only |
| | from EBV negative (IgG or IgM) donors. |
| 12 | Invasive aspergillus, histoplasmosis, or coccidioidomycosis |
| | infection within one year prior to enrollment. |
| 13 | Any history of malignancy except for completely resected |
| | squamous or basal cell carcinoma of the skin. |
| 14 | Known active alcohol or substance abuse. |
| 15 | Baseline Hb below the lower limits of normal at the local |
| | laboratory for patients initially enrolled. After initiation of |
| | immunosuppression serum Hb should be not lower than 10 g/L. |
| 16 | Severe co-existing cardiac disease, characterized by any one of |
| | these conditions: |
| | Recent myocardial infarction (within past 6 months). |
| | Left ventricular ejection fraction <30%. |
| | Uncontrolled coronary artery disease. |
| | Known hypercoagulative state or |
| | International Normalized Ratio >1.8 |
| 17 | Uncontrolled hyperlipidemia (fasting LDL cholesterol >130 |
| | mg/dL and/or fasting triglycerides >200 mg/dL) at the time of |
| | enrollment. |
| 18 | Persistent elevation of liver function tests at the time of |
| | enrollment. Persistent serum glutamic-oxaloacetic transaminase |
| | (SGOT [AST]), serum glutamate pyruvate transaminase (SGPT |
| | [ALT]), Alkaline Phosphatase or total bilirubin, with values >1.5 |
| | times normal upper limits will exclude a patient. However, |
| | patients with Gilbert's syndrome (elevated unconjugated bilirubin |
| | levels in the absence of any evidence of hepatic or biliary tract |
| | disease) are not excluded. |
| 19 | Severe unremitting diarrhea, vomiting or other gastrointestinal |
| | disorders potentially interfering with the ability to absorb oral |
| | medications (e.g., untreated celiac disease). |
| 20 | Untreated Graves’ disease. |
| 21 | Portal hypertension. |
| 22 | Receiving treatment for a medical condition requiring chronic use |
| | of systemic steroids, except for the use of ≤5 mg prednisone daily, |
| | or an equivalent dose of hydrocortisone, for physiological |
| | replacement only. |
| 23 | Treatment with any anti-diabetic medication other than insulin |
| | within 4 weeks of transplant, apart from the GLP-1 agonists (e.g. |
| | exenatide or liraglutide) which will be discontinued at least 2 |
| | weeks prior to transplant. |
| 24 | Use of any investigational agents within 4 weeks of enrollment. |
| 25 | Administration of live attenuated vaccine(s) within 2 months of |
| | enrollment. |
| 26 | Any medical condition that will interfere with treatment. |
| 27 | Treatment with any immunosuppressive regimen at the time of |
| | enrollment. |
| 28 | A previous islet transplant. |
| 29 | A previous pancreas transplant, unless the graft failed within 3 |
| | months due to thrombosis, followed by pancreatectomy and the |
| | transplant occurred more than 6 months prior to enrollment. |
| 30 | Known allergy or hypersensitivity to polypropylene, PTFE, or |
| | RTV silicone. |
| 31 | Islets from non-heart beating donors will be excluded as well as |
| | from CDC high-risk donors. |
| 32 | Presence of colostomy/ileostomy, incisional hernia or other |
| | deformity of the abdominal wall precluding implantation of the |
| | CP-device. |
| 33 | History of malignant hypertension or other conditions precluding |
| | general anesthesia. |
| 34 | Use of coumadin or other anticoagulant therapy (except aspirin) |
| | or subject with PT-INR >1.5. |

### CP-Device

The CP-device used for treatment is a sterile, non-degradable polymer subcutaneous retrievable device for transplantation of therapeutic cells. Once implanted, the device incorporates with tissue and microvessels around removable plugs, which once removed, form void spaces to support transplanted therapeutic cells. The CP-device is scalable to accommodate various transplant volumes of therapeutic cells. The CP-device is made from biocompatible medical grade polymers suitable for long-term implantation in the body.

The CP-device comprises a mesh scaffold formed into a series of cylindrical chambers. The pores of the mesh are large enough to allow tissue and blood vessels to enter, unlike alternative devices designed to limit immune cell infiltration. Polymer plugs are placed temporarily in the device chambers during development of tissue and microvessels within the pores of the mesh to the circumference of the plugs. The plugs are removed and replaced with the therapeutic cell transplant. The plugs have a smooth surface preventing tissue adhesion, allowing easy removal without damaging adjacent microvessels. The resultant tissue chambers that form are of a diameter that is suitable for therapeutic cell transplantation.

There is an open end of the CP-device, through which the therapeutic cells are transplanted. The closure zone at the open end is sutured, closing the CP-device and preventing leakage of cells. A schematic diagram of an exemplary CP-device used for treatment is shown in FIG. 12.

### Treatment Regimens

### CP-Device System

Two to four CP-devices are implanted in the abdomen at two different time points for a maximum of six transplanted CP-devices. Beginning on the day of implant and day of transplant, patients receive Cephazolin intravenously (IV) at the time of the procedure and continue orally with Keflex for 24 hours after CP-device implantation and 7 days after islet infusion (or alternative antibiotic in case of allergy). Subsequent changes in dose and/or medications are made as clinically necessary. Islets are transplanted between approximately 4-24 weeks following CP-device implantation. This timeframe may allow for factors such as access to organ donors for rarer blood groups combined with successful islet cell isolations to be accommodated.

The islet graft is assessed against final islet product release criteria. In addition, safety monitoring of additional sets of CP-devices follow the same timeline as the initial set of implanted CP-devices.

A mini CP-device is implanted at the same time as each set of CP-device implantations and is transplanted with islets at each CP-device islet transplant. The mini CP-device is removed for histological analysis of the islet graft approximately 90±5 days following islet transplantation in each case.

### Immunosuppression

Each patient receives induction therapy with thymoglobulin (ATG) in the standard dose most commonly used in kidney and/or pancreas transplantation. A total dose of 6 mg/kg is divided in at least 4 daily infusions, depending on how patient tolerates the therapy. Subjects receive pre-medication consisting of acetaminophen, diphenhydramine, and hydrocortisone. Intensive insulin treatment is implemented for optimal glucose control during steroid administration. At the same time, maintenance immunosuppression is initiated, consisting of tacrolimus and mycophenolate mofetil (MMF) 500 mg twice a day (BID) or mycophenolic acid (MPA) 360 mg BID. The tacrolimus dose is adjusted daily to gradually achieve level of 4-6 ng/ml. Immunosuppression is initiated a minimum of 3-4 weeks after CP-device implantation in order not to affect its engraftment.

In the case of intolerability, medications can be replaced with an alternative according to the clinical practice. Patients are activated on the United Network for Organ Sharing (UNOS) waiting list for islet transplant 3 weeks later, once stable concentration of the immunosuppressive medication is achieved and cytokine storm related to thymoglobulin therapy is resolved, to create optimal conditions for islet transplantation and engraftment.

On day-1 or day 0 in relation to islet transplant, tacrolimus dose is adjusted to achieve blood level of 8-10 mg/ml, and MMF dose is increased to 1000 mg BID, alternatively MPA to 720 mg BID. On day 0 (prior to islet infusion), patients also receive anti-inflammatory therapy with etanercept (targeting TNFα) at a dose of 50 mg IV followed by 50 mg SC on days 2, 4, and 7. During subsequent islet transplant, basiliximab (targeting IL2 receptor) replaces thymoglobulin for induction therapy.

### Pancreas Procurement and Islet Preparation

Pancreas procurement and preservation, as well as islet isolation and culture, are carried out in accordance with approved protocols. A sample aliquot of islets is taken just prior to transplant for histological assessment.

Release Criteria for Islet Transplantation into CP-Device:
Final Islet Product Criteria and testing required:
   Viability at least 80%
   Purity >70%
   Microbial testing:
      Rapid microbial testing [(e.g., Gram stain if the results of the culture-based sterility test are not available prior to transplantation]—No organisms seen
      Sterility (culture-based assay)—No growth over 28 days (results not needed for release of final product)
   Endotoxin [≤5.0 EU/kg of recipient's body weight (total EU/infusion)]
   Mycoplasma testing (must be completed regardless of islet culture time)—Negative result
   Pellet volume <10.5 ml
   Islet count after islet isolation ≥3000 IEQ/kg
   Actual islets infused (after culture, up to 72 hrs) ≥3000 IEQ/kg

Release Criteria for Islet Intraportal Islet Transplantation:
Purity >20%
Pellet volume <15 ml (7.5 ml per infusion bag)
Islet count after islet isolation ≥5000 IEQ/kg
Actual islets infused (after culture, up to 72 hrs) ≥4000 IEQ/kg

Islet Dosage and Route

For infusion of islets into the CP-device (up to 2 transplants per patient), a minimum target of highly purified islets (≥3000 IEQ/kg recipient body weight at the time of transplant) are transplanted into the CP-device under general or local anesthesia. Islets may be maintained in supplemented CMRL1066-based culture media until the time of transplant, after which they are re-suspended in the patient's own plasma. The spun islet tissue volume preparation is approximately 7.0 or 4.6 cc (3.5 or 2.3 cc/CP-device×2devices), in order to match the capacity of the 10 plug CP-device or 8 plug CP-device, respectively.

For intraportal infusion, the minimal islet dose is ≥5000 IEQ/kg of recipient body weight at the time of completion of isolation. Intraportal infusion and follow up is conducted according to approved protocols.

### Safety and Efficacy Endpoints

### Primary Endpoint

The primary endpoint is a safety endpoint based on following. Safety of the CP-device is assessed following each initial CP-device implantation, at the time of each islet transplantation and following islet transplantation, and at 90±5 days post-islet transplant into the CP-device. Safety is further assessed at approximately 30, 180, 270±5 days, and 365 +14 days post-islet transplant into the CP-device, and at 75±5 and 365±14 days after islet intraportal infusion. A safety assessment is completed at each endpoint time. In addition, safety is continually monitored and assessed throughout treatment. Safety is assessed by evaluating the incidence and severity of adverse events (AEs) determined to be probable or highly probable to the CP-device.

An adverse event (AE) is defined as any untoward medical occurrence in a patient or clinical investigation subject administered a pharmaceutical product or medical device, which does not necessarily have a causal relationship with this treatment. An AE can therefore be any unfavorable and unintended sign (including an abnormal laboratory finding), symptom, or disease temporally associated with the use of a medicinal (investigational) product, whether or not related to the medicinal (investigational) product.

### Secondary Endpoints

The secondary endpoints are focused on efficacy of the islet transplantation into the CP-device, where the day of transplant is designated day 0:
1. Survival of endocrine tissue in the CP-device (defined by positive staining of islets during histological analysis), as evaluated at day 90±5 days (post-removal of mini CP-device); or post-removal of CP-devices at any time during treatment.
2. The proportion of subjects with a reduction in severe hypoglycemic events from day 0 to 90±5 days; 90±5 days to 180±5 days; 180±5 days to 365±14 days following final CP-device transplant and/or last islet transplant.
3. The proportion of subjects with a reduction in HbA1c >1 mg % in the time frame from day 0 to 90±5 days; 90±5 days to 180±5 days; 180±5 days to 365±14 days following each CP-device transplant and/or last islet transplant.

### Results

### Efficacy

Exemplary data from the first treated patient who received a CP-device islet transplant (Patient #1) are set forth in Table 3. At 90 days post-transplant, a glucose tolerance test was administered to the patient (i.e., the patient was given a high sugar drink) over several hours. The patient showed an increase in blood levels of C-peptide, as well as an increase in blood levels of insulin. Without being bound by theory, C-peptide measured in the bloodstream may be used as a biomarker of insulin distribution to the patient and is generally used as a measure of insulin production by islet cells. C-peptide is typically measured following overnight fasting (fasting C-peptide) and during a glucose tolerance test (glucose-stimulated C-peptide). Together these measures may provide an index of the patient's ability to control blood glucose through the production of insulin.

In the post-transplant assessment, C-peptide was used as a biomarker of transplanted islet insulin production. Enduring C-peptide levels in the patient's bloodstream were observed post-transplant, following stimulation with a meal and also when the patient was fasting.

TABLE 3
Baseline and Post-Transplant Data (Patient #1)

| Islet Transplant Status: | Before | 3 Mo. After |
|---|---|---|
| Bodyweight | 83 kg | 73 kg |
| Hemoglobin A1C | 6.5 | 5.6 |
| Daily Use Of Long Acting Insulin Tresiba | 14 U | 8 U |
| Daily Use Of Short Acting Insulin | 15-16 | 14-15 |
| Severe Hypoglycemic Events | 4 per week | 1 per week |

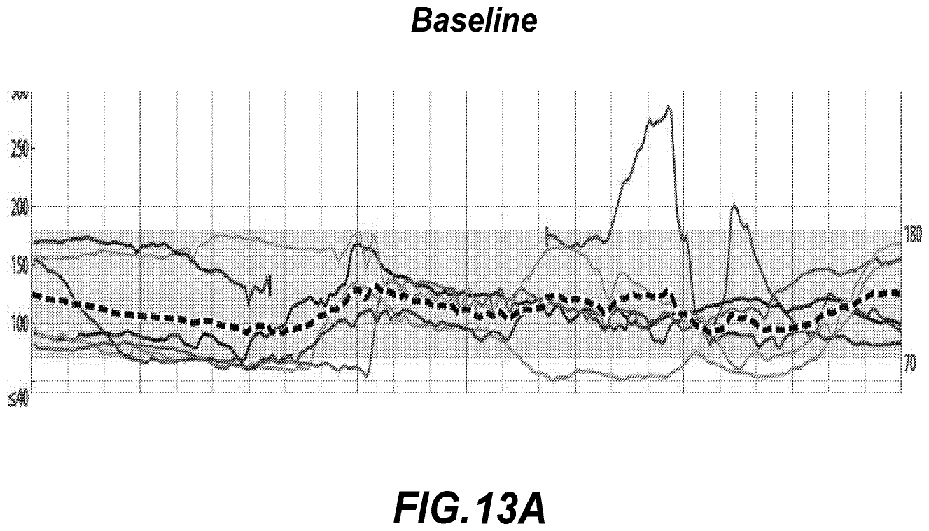

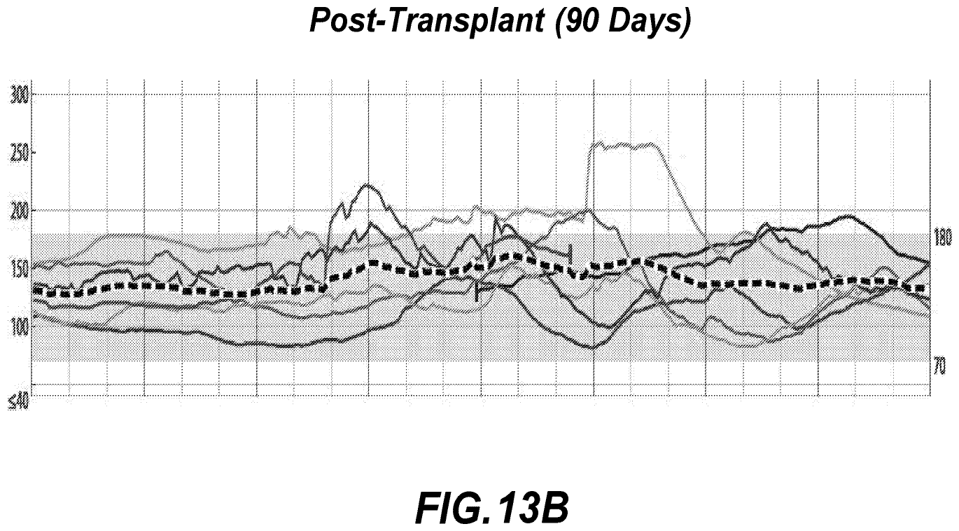

To evaluate blood glucose control before and after islet transplant, including incidents of hypoglycemia and hyperglycemia, Continuous Glucose Monitoring (CGM) was performed. Blood glucose levels over time (mg/dL) are shown in FIG. 13A (baseline) and FIG. 13B (90 days post-transplant). Parameters evaluated at baseline and at 90 days post-transplant are set forth in Table 4. At baseline CGM, the following was observed: more excursions, more hyper/hypo events, and less time in range (time above 180 mg/dL=9%; time in range of 70-180 mg/dL=79%; time below 70 mg/dl=12%). In contrast, at CGM post-CP-device islet transplant, the following was observed: fewer excursions, fewer hyper/hypo events, and more time in range (time above 180 mg/dL=8%; time in range of 70-180 mg/dl=91%; time below 70 mg/dL=1%). These observations suggest improved blood glucose control and stabilization of fluctuating blood sugar levels commonly experienced in patients with type 1 diabetes. This normalizing response to the body's varied need for insulin production may also decrease the likelihood of life threatening hypoglycemic unaware events.

TABLE 4
Baseline and Post-Transplant CGM Parameters (Patient #1)

| | | POST- |
|---|---|---|
| | | TRANSPLANT |
| PARAMETER | BASELINE | (90 DAYS) |
| HIGH GLUCOSE VALUE (MG/DL) | 285 | 231 |
| LOW GLUCOSE VALUE (MG/DL) | 50 | 66* |
| # GLUCOSE EXCURSIONS | 15 | 3 |
| # HIGH EXCURSIONS | 7 | 2 |
| # LOW EXCURSIONS | 8 | 1 |
| STANDARD DEVIATION | 37 | 31 |

*Lowest excursion was 66 mg/dL and this occurred only once.

Exemplary initial observations from Patient #1 also included:
No incidences of adverse events determined to be related to CP-device implant.
CP-device well-incorporated with vascularized tissue, which allowed for effective transplant of purified islets.
A weight reduction of 6.35 kg (12% total body weight).
Stabilizing improvements in all glycemic control parameters as indicated by Continuous Glucose Monitoring (CGM).
87.5% reduction in hypoglycemic events from baseline collected over a two-week monitoring period.
Presence of stimulated blood levels of C-peptide and insulin at the observed 90-day post-transplant point as indicated in a mixed meal tolerance test.

### Secondary Endpoint

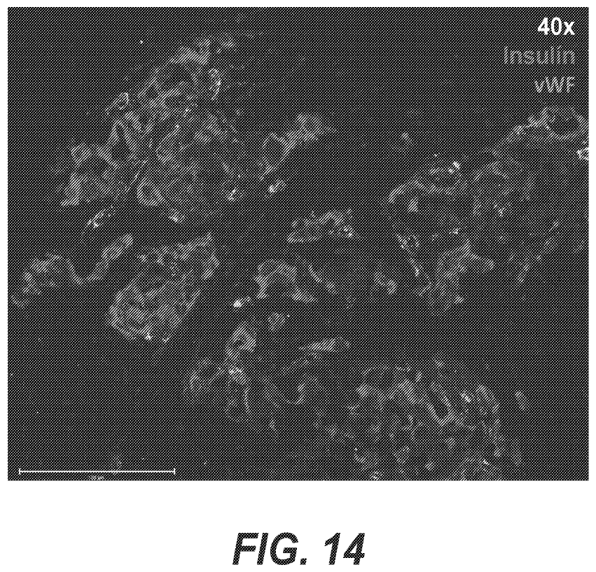

Survival of endocrine tissue (insulin-producing islets) was evaluated at 90 days post-transplant in Patient #1 by measuring positive staining of islets during histological analysis. In Patient #1, explanted mini CP-devices showed abundant viable and organized islet cells intimately associated with blood vessels within a collagen matrix after 90 days of transplantation; the surviving islet cells also strongly expressed insulin (FIG. 14). These observations indicate that the transplanted islet cells within the CP-devices remaining in the patient are healthy, and that these cells have the ability to produce insulin and to deliver insulin to the bloodstream.

Example 2: Imaging of Islets Following Mini CP-Device Removal (Day 90 Post-Transplant)

A mini CP-device and two 8-plug CP-devices were implanted under the skin of a type 1 diabetes patient, as described in Example 1. Islets were subsequently transplanted into the devices. Approximately 90 days post-transplant, 90-day timepoint assessments were completed and the mini CP-device was removed and processed for histology. Results showed the implanted islets were vascularized, actively producing hormones, and delivering C-peptide to the vasculature.

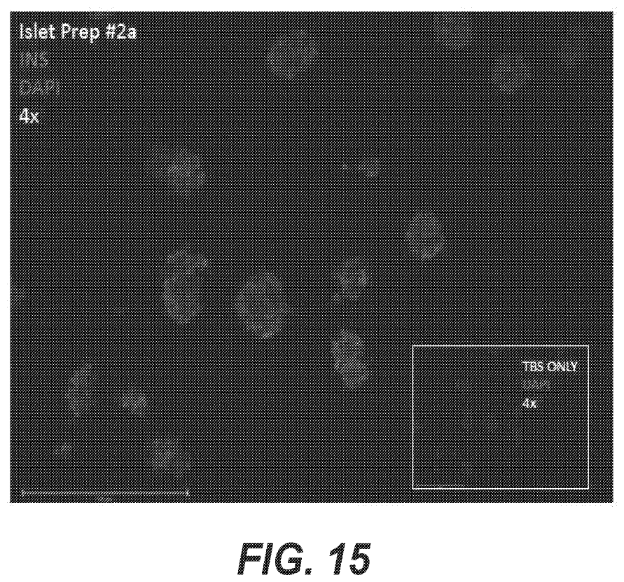

FIG. 15 shows a representative histological image of donor islets transplanted into a CP-device in the patient (5 micron serial sections, paraffin embedded). Immunofluorescence staining for insulin (INS) is shown in red and for cell nuclei (DAPI) in blue. A TBS (Tris-buffered saline) only (no primary antibody) negative control image is also shown.

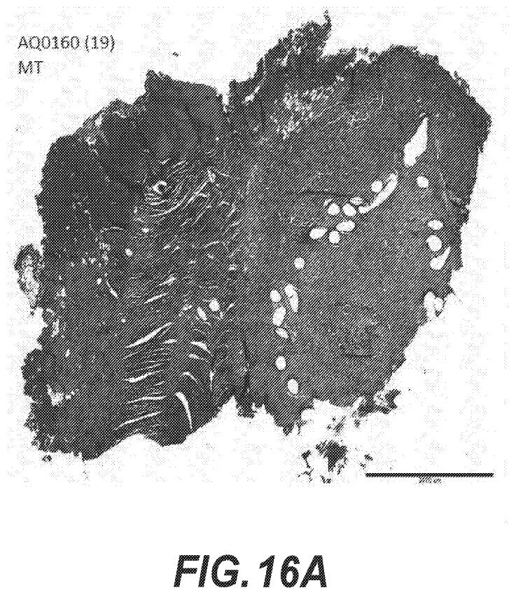

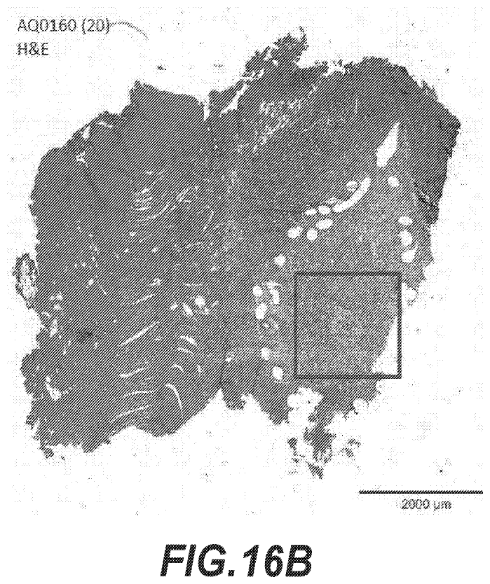

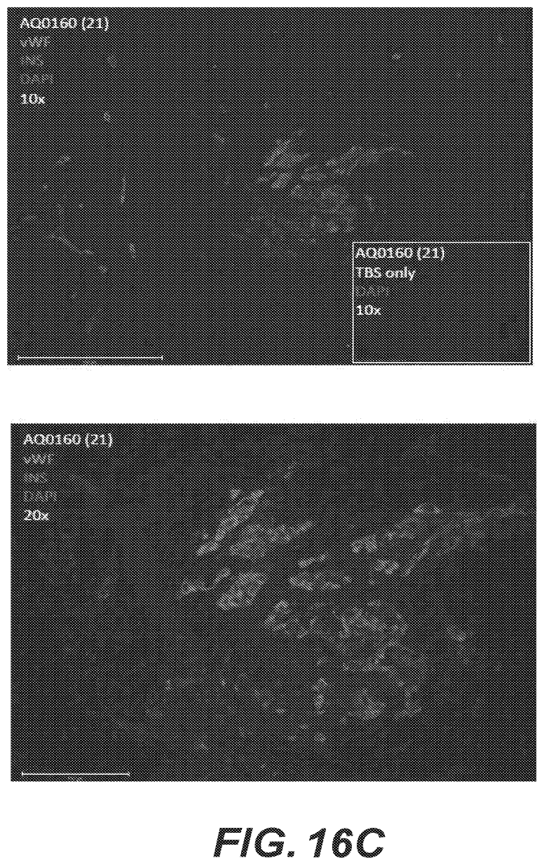

FIG. 16A-16B show representative Masson's Trichrome (MT) (FIG. 16A) and Hematoxylin and Eosin (H&E) (FIG. 16B) scans of a segment (AQ0160 20) from the mini CP-device removed from the patient (5 micron serial sections, paraffin embedded). FIG. 16C shows positive immunofluorescence staining (AQ0160 21) for Insulin (INS, red) and von Willebrand Factor (vWF, green) and cell nuclei (DAPI, blue). A TBS only (no primary antibody) negative control image is also shown.

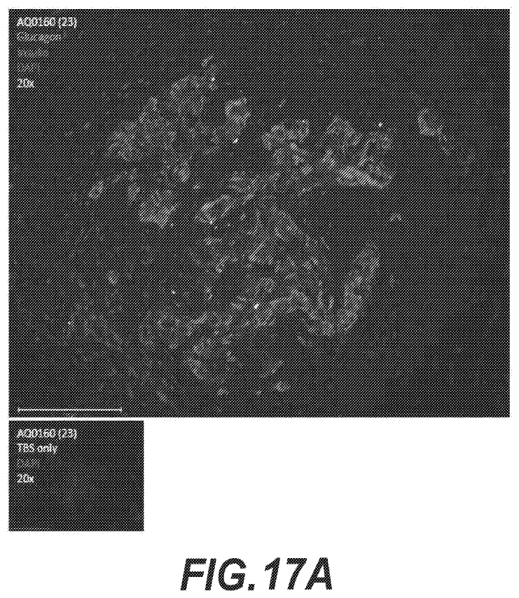

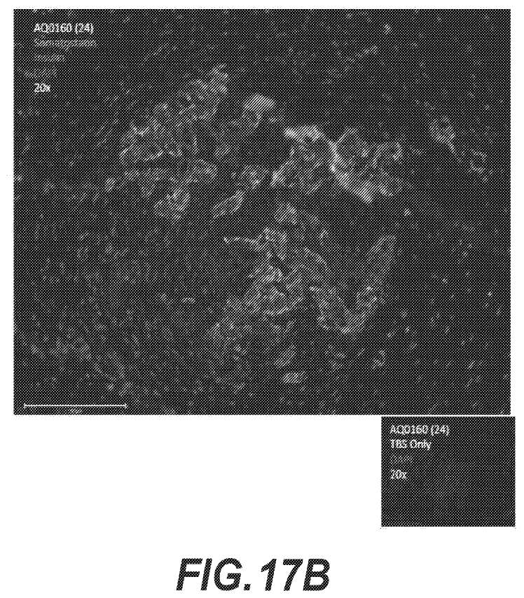

FIG. 17A-17B show representative immunofluorescence images of a segment (AQ0160 23/24) from the mini CP-device removed from the patient (5 micron serial sections, paraffin embedded). FIG. 17A shows positive immunofluorescence staining (AQ0160 23) for Insulin (INS) and Glucagon (GLUC) [Red: Insulin, Green: Glucagon, Blue: DAPI (Nuclei)]. A TBS only (no primary antibody) negative control image is also shown. FIG. 17B shows positive immunofluorescence staining (AQ0160 24) for Insulin (INS) and Somatostatin (SOM) [Red: Insulin, Green: Somatostatin, Blue: DAPI (Nuclei)]. A TBS only (no primary antibody) negative control image is also shown.

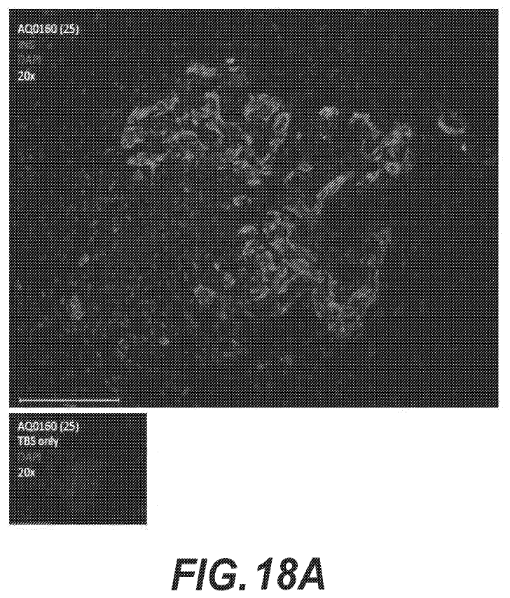

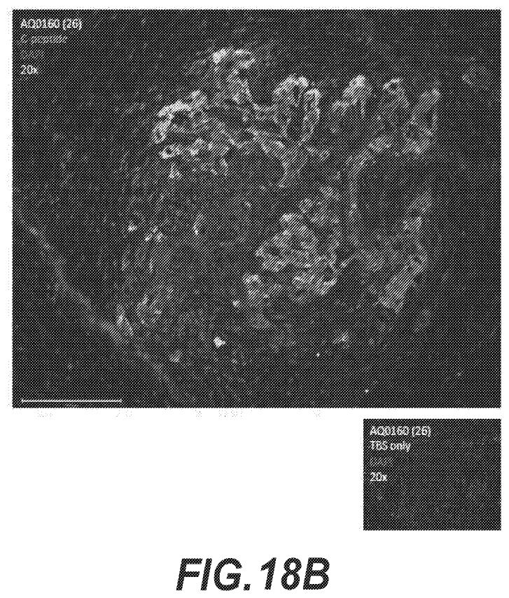

FIG. 18A-18B show representative immunofluorescence images of a segment (AQ0160 25/26) from the mini CP-device removed from the patient (5 micron serial sections, paraffin embedded). FIG. 18A shows positive immunofluorescence staining (AQ0160 25) for Insulin (INS) [Red: Insulin, Blue: DAPI (Nuclei)]. A TBS only (no primary antibody) negative control image is also shown. FIG. 18B shows positive immunofluorescence staining (AQ0160 26) for C-peptide [Green: C-peptide, Blue: DAPI (Nuclei)]. A TBS only (no primary antibody) negative control image is also shown.

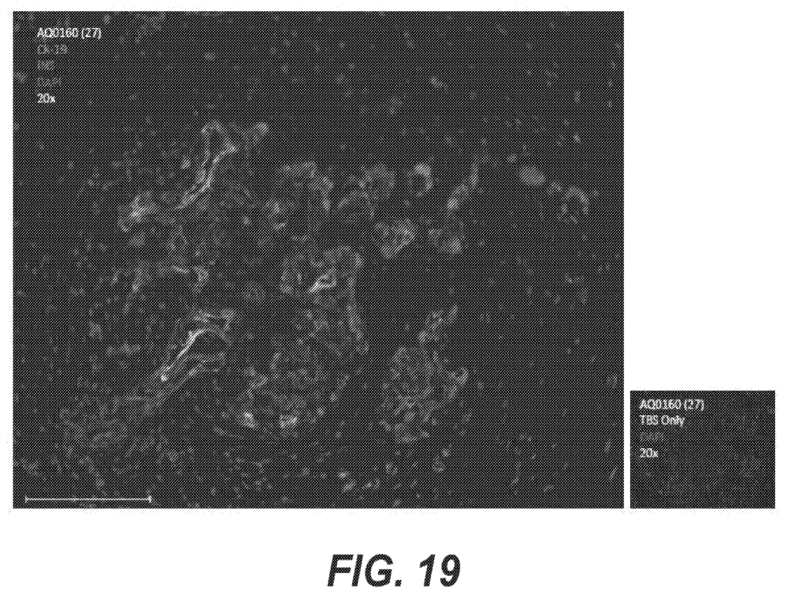

FIG. 19 shows a representative immunofluorescence image of a segment (AQ0160 27) from the mini CP-device removed from the patient (5 micron serial sections, paraffin embedded). Positive immunofluorescence staining (AQ0160 27) for Insulin (INS) and CK 19 is shown [Red: Insulin, Green: CK 19, Blue: DAPI (Nuclei)]. A TBS only (no primary antibody) negative control image is also shown.

1-38. (canceled)

39. An implantable medical device comprising:
(a) a porous scaffold, the porous scaffold comprising an immunologically compatible polymer mesh forming the walls of one or more chambers,
wherein the polymer mesh is selected to allow microvessels to enter the device and be maintained as healthy vessels,
wherein the porous scaffold has pores sized to facilitate growth of vascular and connective tissues around and through the walls of the at least one chamber,
wherein each of the one or more chambers comprises an opening at either or both of a proximal end and a distal end of the chamber,
wherein the proximal end and the distal end are separated by a lumen that is bounded by the walls, and at least one seal configured to enclose either or both of a proximal end and a distal end of the at least one chamber;
(b) at least one removable, non-porous plug configured to be positioned within the lumen each of the one or more chambers,
wherein the plug extends along the lumen of each of the one or more chambers; and
(c) at least one seal configured to enclose either or both the proximal end and the distal end of each of the one or more chambers,
wherein the device is configured to be implanted with insulin producing cells at a borderline mass of about 3,000-5,000 islet equivalent cells per kilogram (IEQ/kg).

40. The implantable medical device according to claim 39, wherein the one or more chambers consists of eight to ten chambers.

41. The implantable medical device according to claim 39, wherein the device further comprises a material coating at least a part of the porous scaffold which stimulates tissue incorporation and angiogenesis.

42. The implantable medical device according to claim 39, wherein
(i) at least about 60%, 65%, 70%, 75%, 80%, 85%, or 90%, e.g., at least 70% of the insulin producing cells are purified islets;
(ii) at least about 70%, 75%, 80%, 85%, or 90%, e.g., at least 80% of the insulin producing cells are viable islets; and/or
(iii) the insulin producing cells are purified from a pellet prior to administration, and the pellet has a volume of less than about 15 mL, e.g., less than about 10.5 mL.

43. The implantable medical device according to claim 39, wherein the device is configured to be implanted with insulin producing cells at a borderline mass of about 3,000-4,000 IEQ/kg.

44. The implantable medical device according to claim 39, wherein the device is configured to be implanted with insulin producing cells at a borderline mass of about 3,000 IEQ/kg.

45. The implantable medical device according to claim 39, wherein the biodegradable material comprises one or more of a growth factor, an antifibrotic agent, a polymer, vascular endothelial growth factor (VEGF), collagen, fibronectin, polyethylene-imine and dextran sulfate, polyvinyl siloxane and polyethylenimine, phosphorylchloride, poly (ethylene glycol), poly (lactic-co-glycolic acid), poly (lactic acid), polyhydroxyvalerate and copolymers, polyhydroxybutyrate and copolymers, polydiaxanone, polyanhydrides, poly (amino acids), poly (orthoesters), gelatin, a cellulose polymer, a chitosan, an alginate, vinculin, agar, agarose, hyaluronic acid, and Matrigel.

46. The implantable medical device according to claim 39, wherein the device further comprises at least one cell infusion tube configured to be positioned within the chamber and configured to deliver insulin-producing cells into the one or more chambers of the device.

47. The implantable medical device according to claim 39, wherein the device is suitable for use in treating diabetes.
